SEQUENCE LISTING (7 sequences)

SEQ ID NO: 1 - DNA, 28 bp, synthetic construct
  source 1..28: mol_type = other DNA
ggtttagtgaaccgtcagatccgctagc

SEQ ID NO: 2 - DNA, 24 bp, synthetic construct
  source 1..24: mol_type = other DNA
tcgatcccgtaccgaggagatctg

SEQ ID NO: 3 - DNA, 30 bp, synthetic construct
  source 1..30: mol_type = other DNA
atttgtctcgaggtcgattcgaatacgcgt

SEQ ID NO: 4 - DNA, 96 bp, synthetic construct
  source 1..96: mol_type = other DNA
cccagaaggcgggagacatagggggagcccacaccagccatcacgtatgccatcacgtatgcttcgtctaagatttctttgttggctttgggggat

SEQ ID NO: 5 - DNA, 12 bp, synthetic construct
  source 1..12: mol_type = other DNA
cgtcagatccgc

SEQ ID NO: 6 - DNA, 12 bp, synthetic construct
  source 1..12: mol_type = other DNA
gcgatcgcagcg

SEQ ID NO: 7 - DNA, 12 bp, synthetic construct
  source 1..12: mol_type = other DNA
cgaggagatctg

(19) United States
(12) Patent Application Publication
(10) Pub. No.: US 2026/0201366 A1
(43) Pub. Date: Jul. 16, 2026

(54) MUTATION EFFECT AND ACTIVITY PROFILE MAPPING SYSTEM AND METHOD

(71) Applicant: Heligenics Inc., Las Vegas, NV (US)

(72) Inventors: Martin Roy SCHILLER, Henderson, NV (US); Elizabeth Joy VALENTE, Henderson, NV (US); Lancer BROWN, Henderson, NV (US); Christopher John GIACOLETTO, Las Vegas, NV (US); Jerome I. ROTTER, Los Angeles, CA (US)

(21) Appl. No.: 19/349,253

(22) Filed: Oct. 3, 2025

Related U.S. Application Data
(63) Continuation of application No. PCT/US2024/023134, filed on Apr. 4, 2024.
(60) Provisional application No. 63/494,228, filed on Apr. 4, 2023.
(60) Provisional application No. 63/498,831, filed on Apr. 28, 2023.

(51) Int. Cl. C12N 15/10

(52) U.S. Cl. CPC C12N 15/1065

(57) ABSTRACT

The method as disclosed herein utilizes a high throughput screening assay (GigaAssay) to produce a comprehensive mutation effect on gene activity (MEGA)-mutation activity profile (Map). The methods as disclosed herein can be used to assess the mutational effect for any gene, under any condition (e.g., drug treatment) with any assay in mammalian cells in culture. Thus, the methods provided herein can be utilized to discover and screen dominant negative variants, and provide a reliable solution to the problem of identifying unique pharmacologically active variants of proteins. Furthermore, the method provided herein can be integrated to cell-based assays to investigate disease pathology and test potential drugs.

### CROSS-REFERENCE

This application is a continuation of PCT/US2024/023134, filed Apr. 4, 2024, which claims the benefit of U.S. Provisional Application No. 63/494,228, filed Apr. 4, 2023, and U.S. Provisional Application No. 63/498,831, filed Apr. 28, 2023, the entire contents of which are incorporated herein by reference in their entireties.

### SEQUENCE LISTING

The instant application contains a Sequence Listing which has been submitted electronically in ST.26 (xml) format and is hereby incorporated by reference in its entirety. Said ST.26 (xml) copy, created on Jun. 10, 2024, is named 217863_706601_xml and is 7,274 bytes in size.

### SUMMARY

Disclosed herein is a method for identifying mutational effects on protein activity, the method comprising: (a) obtaining a library comprising a plurality of cDNAs, wherein each cDNA in the plurality of cDNA encodes a unique variant(s) of a target protein, wherein each unique variant has one or more amino acid substitutions relative to the target protein; (b) independently and individually incorporating one cDNA of the plurality of cDNAs into one plasmid, such that each cDNA of the plurality of cDNAs is individually and independently incorporated into its own plasmid and operably linked to a promoter on the plasmid, forming a plasmid cDNA library, and wherein each plasmid in the plasmid cDNA library further comprises a unique molecular identifier (UMI) or a barcode group; (c) converting the plasmid cDNA library into a viral library comprising virions, wherein each virion comprises only one plasmid of the plasmid DNA library, wherein the viral library can optionally be a lentiviral library; (d) engineering a human or mammalian cell line to encode a receptor, optionally a surface-expressed receptor, operably linked to a reporter system such that the receptor and the reporter system can indicate if one of the unique variant(s) binds to and modulates a biological activity of the receptor, thereby forming an engineered human or mammalian cell line; and (e) transducing cells of the engineered human or mammalian cell line of (d) with virions of the viral library of (c), such that each virion transduces a different cell, thereby forming a library of transduced cells. In some embodiments, the method results in the expression of the unique variant of the target protein in a transduced cell of the engineered or mammalian cell line. In Some embodiments, the method further comprises determining a measurement of a biological activity of the unique variant in the transduced cell of the engineered or mammalian cell line. In some embodiments, the determining comprises calculating with a computer a measurement of the biological activity. In some embodiments, the method further comprises sorting a transduced cell comprising the unique variant using a UMI or a barcode group by flow-cytometry. In some embodiments, the method further comprises sorting a plurality, or all of the cells from the library of transduced cells resulting in the formation of a flow-sorted pool(s) of the UMI(s) or a flow-sorted pool(s) of the barcode group. In some embodiments, the measurement of the biological activity comprises detecting binding of the unique variant(s) to the receptor operably linked the reporter system and determining an amount, an intensity, or both of a report from the reporter system, as measured by fluorescence microscopy, flow cytometry, or both. In some embodiments, the method further comprises comparing a distribution of the flow-sorted pools of the UMIs or the barcode groups of a single cell group that individually express the unique variant(s) to a distribution of flow-sorted pools of a single cell group expressing a wildtype target protein or a wildtype cDNA without the one or more amino acid substitutions. In some embodiments, the measurement of the biological activity of the unique variant(s) is repeated at least 100 times using the UMI, the barcode, or both. In some embodiments, each UMI, or each barcode independently comprises about 12 nucleotides to about 32 nucleotides. In some embodiments, the cells of the engineered human or mammalian cell line are transduced with the viral library at a multiplicity of infection (MOI) of at least 0.1 to at least 1. In some embodiments, the MOI is 0.1. In other embodiments, the MOI prevents double insertions of a virion into a single transduced or minimizes a transduction error rate. In some embodiments, the cells of the engineered human or mammalian cell line are selected with a marker for lentiviral integration. In some embodiments, the receptor and the reporter system are operably linked to an inducible-promoter. In some embodiments, the inducible-promoter drives the expression of the receptor and the reporter system. In some embodiments, the inducible-promoter comprises a doxycycline inducible-promoter. In some embodiments, the reporter system comprises a polynucleotide that encodes a fluorescent protein, and wherein the fluorescent protein is GFP. In some embodiments, a single cell group is isolated based on the UMI using flow cytometry, and sorted into one or more bins. In some embodiments, the one or more bins comprise the cDNA(s) encoding the unique variant(s), an amplicon(s) thereof, or any combination thereof. In some embodiments, the cDNA(s) encoding the unique variant(s), the amplicon(s) thereof, or any combination thereof are sequenced using a sequencing method. In some embodiments, the sequencing method comprises next-generation sequencing, Sanger sequencing, whole genome sequencing, RNA sequencing or shotgun sequencing. In some embodiments, the sequencing method is a next-generation sequencing, and wherein the next-generation sequencing results in a sequence dataset. In some embodiments, the sequence data comprises a read depth for each unique variant. In some embodiments, each unique variant has a read depth of about 2000× to about 90,000× sequencing coverage. In some embodiments, the single cell group expressing a unique variant is compared to the single cell group expressing a wildtype target protein with a statistical model to test a specific hypothesis regarding the biological activity of each unique variant. In some embodiments, the biological activity of each unique variant comprises a loss-of-function variant, a gain-of-function variant, a variant with substantially similar activity to the wildtype target protein, a drug resistant variant, or a drug susceptible variant. In some embodiments, the method further comprises validating the method by comparing the biological activity of a subset of unique variants analyzed by the method to a previously determined activity of a wildtype target protein. In some embodiments, the method further comprises validating the method by comparing true negatives as determined by the method to true negatives determined by an independent method. In some embodiments, the method further comprises validating the method by comparing the biological activity of a subset of unique variants analyzed by the method to an independent test of a set of separate clones comprising the unique variant(s). In some embodiments, the method further comprises validating the method by comparing the method results among one or more different samples. In some embodiments, the method further comprises validating the method by comparing the method results in two different cell lines. In some embodiments, the method further comprises generating an oligonucleotide sequence dataset. In some embodiments, the method further comprises analyzing the oligonucleotide sequence dataset using a computer, wherein the analyzing comprising: (a) generating a unique variant-UMI index library using long reads from next-generation sequencing (NGS); (b) identifying UMI counts and barcode bins by analyzing short reads of flow-sorted groups; (c) calculating an activity score from a UMI-barcode read counts comprising the UMI counts and barcode bins of (b); (d) assessing an effect of the unique variant(s) on biological activity by calculating read percent of the reporter protein, such as a fluorescent protein, and p-value; and (e) accurately and quantitatively assessing activities of the unique variant(s) with respect to previously characterized wildtype, wherein the fluorescent protein is GFP, and wherein biological activity comprises a gene activity or a protein activity. In some embodiments, the library of transduced cells comprises from about 10,000 to about 10 million cells. In some embodiments, the library of transduced cells comprises about 10,000 cells, 20,000 cells, 30,000 cells, 40,000 cells, 50,000 cells, 60,000 cells, 70,000 cells, 80,000 cells, 90,000 cells, 100,000 cells, 150,000 cells, 200,000 cells, 300,000 cells, 400,000 cells, 500,000 cells, 600,000 cells, 700,000 cells, 800,000 cells, 900,000 cells, 1,000,000 cells, 2,000,000 cells, 3,000,000 cells, 4,000,000 cells, 5,000,000 cells, 6,000,000 cells, 7,000,000 cells, 8,000,000 cells, 9,000,000 cells or 10,000,000 cells.

Also provided herein is a protein variant discovered by the method as disclosed herein.

Provided herein is a library of transduced cells formed by the method as disclosed herein. In some embodiments, the library of transduced cells comprises from about 10,000 cells to about 10 million cells.

Also provided herein is a library of isolated and purified transduced cells comprising from about 10,000 cells to about 10 million cells, wherein each isolated and purified transduced cell contains a plasmid comprising a cDNA that encodes for a unique variant of a wildtype protein, such that the transduced cell further comprises a surface-expressed receptor operably coupled to a reporter system, and wherein the cell-surface receptor is capable of interrogating each unique variant. In some embodiments, each isolated and purified transduced cell further comprises on its surface a unique variant.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present disclosure will be obtained by reference to the following detailed description that sets forth illustrative embodiments, in which the principles of the present disclosure are utilized, and the accompanying drawings of which:

FIG. 1A-1D depicts a schematic for generation of a gene mutation library (GML) analyzed with the high throughput screening assay as disclosed herein (GigaAssay) to produce a comprehensive mutation effect on gene activity (MEGA)-mutation activity profile (Map), combined to (MEGA-Map). FIG. 1A depicts the steps for creating a gene mutation library as disclosed herein. FIG. 1B depicts the steps for validating the library as disclosed herein. FIG. 1C depicts the high throughput assay as disclosed herein. FIG. 1D depicts the steps for the bioinformatics pipeline.

FIG. 2 depicts variant assembly. FIG. 2 shows (from left to right) the 5′LTR, TreTIGHTpro, the gene of interest (GOI) coding sequence containing a variant region that corresponds to 50 amino acids of the coding sequence in the context of a lentiviral expression vector, the GOI is followed by AsiSI-Mlul restriction sites in the 3′UTR which corresponds to the Unique Molecular Identifier (UMI) insertion site which, followed by the 3′LTR.

FIG. 3 depicts variant assembly. FIG. 3 shows zoomed in depiction of the variant region in the context of dsDNA. The variant region is 150 bp and 25 bp of overlapping DNA sequence flanking the variant region results in 1 tile of 200 bp.

FIG. 4 depicts comparison of inverse PCR product and the variant oligo pool in preparation for HiFi DNA Assembly. Inverse PCR primers (arrows) are designed so they include the 25 bp of sequence overlap with the oligo pool. The oligo pool consists of up to thousands of oligos containing desired mutations (asterisks).

FIG. 5 depicts T5 Exonuclease activity during HiFi DNA Assembly. T5 Exonuclease chews back dsDNA strands in 5′->3′ direction. This exposes 3′ DNA strands for annealing to the complementary sequence found in the oligo pool. Note: This example is focusing on 1 oligo from the oligo pool.

FIG. 6 depicts HiFi DNA Polymerase and DNA Ligase activity during HiFi DNA Assembly. The HiFi DNA Polymerase synthesizes DNA in a 5′->3′ direction, synthesizing the reverse strand of the variant oligo as well as filling in any gaps that occur after the annealing of DNA strands. DNA Ligase then seals the nicks to create a continuous DNA strand. Note: This example is focusing on 1 oligo from the oligo pool.

FIG. 7 depicts continuation of T5 Exonuclease activity during HiFi DNA Assembly. T5 Exonuclease continues to digest exposed ends of DNA strands from 5′->3′ direction, exposing DNA for annealing complementary strands. Note: This example is focusing on 1 oligo from the oligo pool.

FIG. 8 depicts continuation of HiFi DNA Polymerase and DNA Ligase during HiFi DNA Assembly. The DNA Polymerase continues to synthesize DNA in 5′-3′ direction to fill in gaps, and DNA Ligase seals nicks to create continuous strands of DNA. The final result of a complete assembly is a scarless recombinant vector containing the variants of interest cloned into the variant region. Note: This example is focusing on 1 oligo from the oligo pool.

FIG. 9 depicts the final product of HiFi DNA Assembly. The result of a complete assembly is a scarless recombinant vector containing the variants of interest cloned into the variant region. This exemplary HiFi DNA assembly focuses on 1 oligo from the oligo pool resulting in 1 variant combination.

FIG. 10 depicts the target DNA Sequence is the variant region that is being targeted for the gene mutation library. The target DNA sequence is divided into tiles of 200 nucleotides. These tiles will correspond to 200-mer synthetic oligos for the variants of interest. Each tile consists of a variant region (yellow) flanked by 25 nucleotides that overlap with the vector cloning site which also serve as Inverse PCR Primer binding sites for inverse PCR of the vector DNA. The tiles will overlap so that the entire target region is covered. In this example the target DNA sequence is 600 bp long, covered by 3 overlapping tiles. Each tile will be treated as separate Variant Assembly reactions into the expression vector using the same general strategy as described above. Variant Assembly will result in 3 separate assembly product pools which will be combined to generate the plasmid variant library (without UMI). There are unique restriction sites in the 3′UTR of the vector (AsiSI and Mlul). These sites will be used to linearize the vector DNA to allow for HiFi DNA assembly to add a UMI to the 3′UTR with a ssDNA UMI oligo, resulting in a plasmid variant library with UMIs added to each plasmid DNA molecule of the library.

FIG. 11 depicts a zoomed in on the 3′UTR UMI Assembly region looking at it in context of dsDNA. In this example AsiSI and MluI are the restriction sites that will be used to linearize the variant plasmid library. Thick dashed lines indicate cleavage sites.

FIG. 12 depicts comparison of vector DNA linearized with AsiSI-Mlu and UMI Oligo. The HiFi DNA Assembly reaction is performed using AsiSI-MluI linearized vector DNA and a UMI oligo that has 25 nucleotides of overlapping sequence with the cloning site. The assembly reaction is carried out similar to FIGS. 4-7 (above) until assembly is complete.

FIG. 13 depicts an exemplary completed plasmid gene mutation library. The final product of the UMI Assembly is a plasmid GML containing variants of interest with a unique molecular identifier for each plasmid DNA molecule in the library.

FIG. 14 depicts a Bioinformatics Pipeline flowchart. The goal of this process is to evaluate the performance of mutants identified and created by the high throughput screening assay (GigaAssay) as disclosed herein during a performance screening on a GML that requires long read sequencing identification of the library resulting in a sequence dataset. In order to analyze the sequence dataset containing the outputd of the sorted pools, unique variants must first be identified and mapped to their 32 BP barcode bins. After identification, the barcodes bins are quantified and their representation in each of the sorted pools is analyzed. This allows interpretation of the results of the method as disclosed herein.

FIG. 15 depicts a design of a dominant negative GML library for candidate genes prioritized by GWAS, polygenic risk scores (PRS) scores, or other Omics approaches.

FIG. 16 depicts a design of a GML library for chimeric genes including a dominant negative version of one candidate genes prioritized by GWAS, polygenic risk scores (PRS) scores, or other Omics approaches.

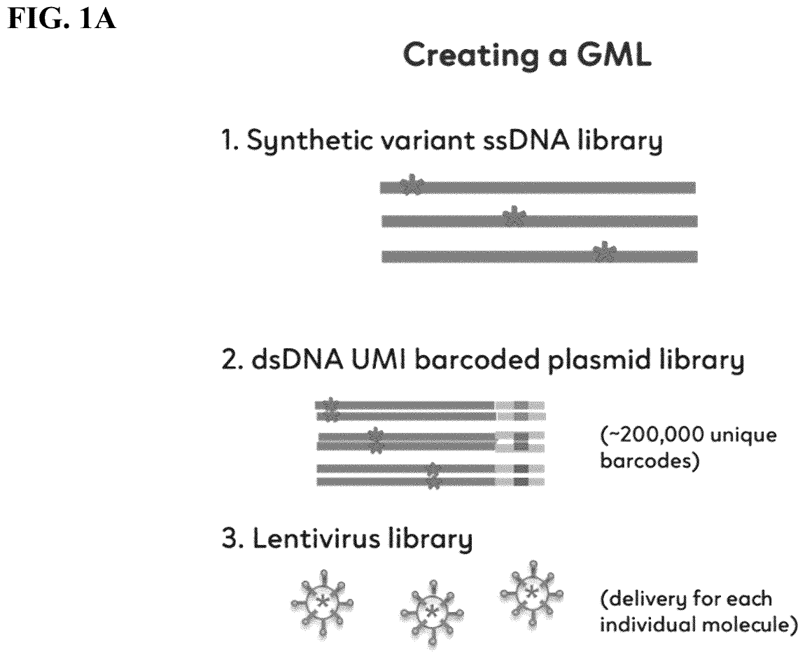

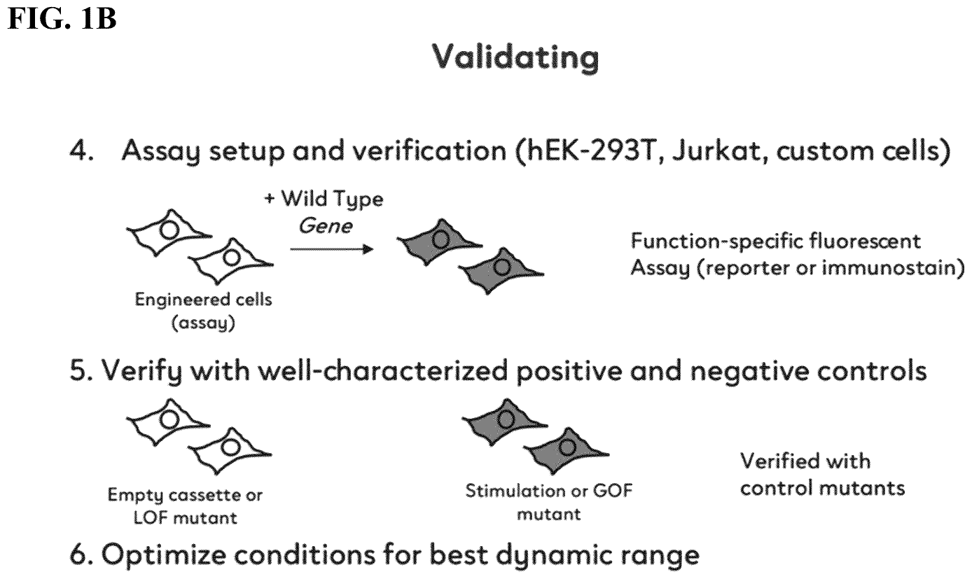

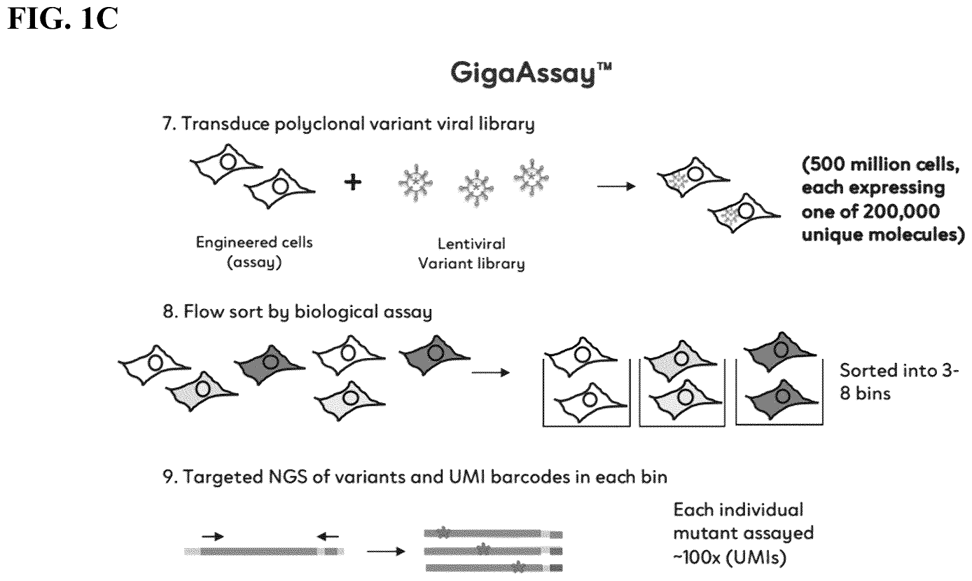

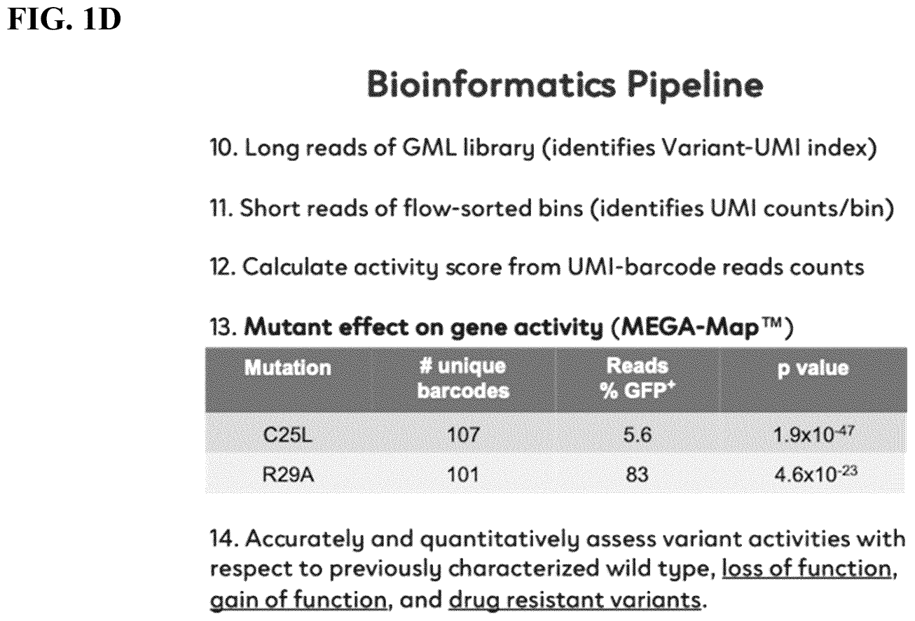

### DETAILED DESCRIPTION OF THE INVENTION

### Overview

The method as disclosed herein utilizes a high throughput screening assay (GigaAssay) to produce a comprehensive mutation effect on gene activity (MEGA)-mutation activity profile (Map). There is no high-throughput assay to broadly assess molecular functions in the context of human or mammalian cells. The molecular function are the key to understanding mechanism, disease etiology, and development of therapeutic drugs. The methods as disclosed herein can be used to assess the mutational effect for any gene, under any condition (e.g., drug treatment) with any assay in mammalian cells in culture. Phage or yeast display, yeast 1- or 2-hybrid, DNA encoded libraries (DEL) s, and affinity mass spectrometry assess one general type of function, molecular interactions, although these do not assess interactions in live mammalian cells. The methods provided herein can be utilized to discover and screen dominant negative variants. The methods provided herein provide a reliable solution to the problem of identifying unique pharmacologically active variants. Here, the methods described herein detail an approach that can be used to prioritize lead protein variants by using the described GigaAssay approach to address this problem. Furthermore, the method provided herein can be integrated to cell-based assays to investigate disease pathology and test potential drugs.

### Definitions

Unless defined otherwise, all technical and scientific terms used herein have the same meaning as commonly understood by one of ordinary skill in the art to which the method of the present disclosure pertains. Although any methods and materials similar or equivalent to those described herein can be used in the practice for testing of the present disclosure, the preferred materials and methods are described herein. In describing and claiming the present disclosure, the following terminology will be used.

It is also to be understood that the terminology used herein is for the purpose of describing particular embodiments only, and is not intended to be limiting. The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of +20% or +10%, more preferably +5%, even more preferably +1%, and still more preferably +0.1% from the specified value, as such variations are appropriate to perform the disclosed methods.

A “biomolecule” or “biological molecule” refers to a molecule that is generally found in or produced by a biological organism. In some embodiments, biological molecules comprise polymeric biological macromolecules having multiple subunits (i.e., “biopolymers”). Typical biomolecules include proteins, enzymes, and other polypeptides, DNA, RNA and other polynucleotides, and can also include molecules that share some structural features with naturally occurring polymers such as RNAs (formed from nucleotide subunits), DNAs (formed from nucleotide subunits), and peptides or polypeptides (formed from amino acid subunits), including, e.g., RNA analogues, DNA analogues, polypeptide analogues, peptide nucleic acids (PNAs), combinations of RNA and DNA (e.g., chimeraplasts), or the like. It is not intended that biomolecules be limited to any particular molecule, as any suitable biological molecule finds use in the present disclosure, including but not limited to, e.g., lipids, carbohydrates, or other organic molecules that are made by one or more genetically encodable molecules (e.g., one or more enzymes or enzyme pathways) or the like.

The terms “unique variant” or “unique variant(s)” refers to a protein that comprise one or more amino acid substitutions as compared to a wildtype protein. Of particular interest for some embodiments of this disclosure are biomolecules having active sites that interact with a reporter molecule (e.g., a receptor) to affect a chemical or biological transformation, e.g., catalysis of a substrate, activation of biomolecules, or inactivation of the biomolecules, specifically enzymes.

In some embodiments, a “biological activity” or “activity” is an increase or decrease in one or more of the following: catalytic rate (k_{cat}), substrate binding affinity (K_{M}/K_{D}), catalytic efficiency (k_{cat}/K_{M}), substrate specificity, chemoselectivity, regioselectivity, stereoselectivity, stereospecificity, ligand specificity, receptor agonism, receptor antagonism, conversion of a cofactor, oxygen stability, protein expression level, solubility, thermoactivity, thermostability, pH activity, pH stability (e.g., at alkaline or acidic pH), glucose inhibition, and/or resistance to inhibitors (e.g., acetic acid, lectins, tannic acids, and phenolic compounds) and proteases. Other desired activities may include an altered profile in response to a particular stimulus (e.g., altered temperature and/or pH profiles). In the context of rational ligand design, optimization of targeted covalent inhibition (TCI) is a type of activity. In some embodiments, two or more variants screened as described herein act on the same substrate but differ with respect to one or more of the following activities: rate of product formation, percent conversion of a substrate to a product, selectivity, and/or percent conversion of a cofactor. It is not intended that the present disclosure be limited to any particular beneficial property and/or desired activity.

In some embodiments, “activity” is used to describe the more limited concept of an enzyme's ability to catalyze the turnover of a substrate to a product. A related enzyme characteristic is its “selectivity” for a particular product such as an enantiomer or regioselective product. The broad definition of “activity” presented herein includes selectivity, although conventionally selectivity is sometimes viewed as distinct from enzyme activity.

“Encoding” refers to the inherent property of specific sequences of nucleotides in a polynucleotide, such as a gene, a cDNA, or an mRNA, to serve as templates for synthesis of other polymers and macromolecules in biological processes having either a defined sequence of nucleotides (i.e., rRNA, tRNA and mRNA) or a defined sequence of amino acids and the biological properties resulting therefrom. Thus, a gene encodes a protein if transcription and translation of mRNA corresponding to that gene produces the protein in a cell or other biological system. Both the coding strand, the nucleotide sequence of which is identical to the mRNA sequence and is usually provided in sequence listings, and the non-coding strand, used as the template for transcription of a gene or cDNA, can be referred to as encoding the protein or other product of that gene or cDNA.

As used herein “endogenous” refers to any material from or produced inside an organism, cell, tissue or system.

As used herein, the term “exogenous” refers to any material introduced from or produced outside an organism, cell, tissue or system.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., Sendai viruses, lentiviruses, retroviruses, adenoviruses, and adeno-associated viruses) that incorporate the recombinant polynucleotide.

“Homologous” as used herein, refers to the subunit sequence identity between two polymeric molecules, e.g, between two nucleic acid molecules, such as, two DNA molecules or two RNA molecules, or between two polypeptide molecules. When a subunit position in both of the two molecules is occupied by the same monomeric subunit; e.g, if a position in each of two DNA molecules is occupied by adenine, then they are homologous at that position. The homology between two sequences is a direct function of the number of matching or homologous positions; e.g, if half (e.g, five positions in a polymer ten subunits in length) of the positions in two sequences are homologous, the two sequences are 50% homologous; if 90% of the positions (e.g, 9 of 10), are matched or homologous, the two sequences are 90% homologous.

In the context of the present disclosure, the following abbreviations for the commonly occurring nucleic acid bases are used. “A” refers to adenosine, “C” refers to cytosine, “G” refers to guanosine, “T” refers to thymidine, and “U” refers to uridine.

Unless otherwise specified, a “nucleotide sequence encoding an amino acid sequence” includes all nucleotide sequences that are degenerate versions of each other and that encode the same amino acid sequence. The phrase nucleotide sequence that encodes a protein or an RNA may also include introns to the extent that the nucleotide sequence encoding the protein may in some versions contain an intron(s).

The term “operably linked” refers to functional linkage between a regulatory sequence and a heterologous nucleic acid sequence resulting in expression of the latter. For example, a first nucleic acid sequence is operably linked with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame. The term “polynucleotide” as used herein is defined as a chain of nucleotides. Furthermore, nucleic acids are polymers of nucleotides. Thus, nucleic acids and polynucleotides as used herein are interchangeable. One skilled in the art has the general knowledge that nucleic acids are polynucleotides, which can be hydrolyzed into the monomeric “nucleotides.” The monomeric nucleotides can be hydrolyzed into nucleosides. As used herein polynucleotides include, but are not limited to, all nucleic acid sequences which are obtained by any means available in the art, including, without limitation, recombinant means, i.e., the cloning of nucleic acid sequences from a recombinant library or a cell genome, using ordinary cloning technology and PCR™, and the like, and by synthetic means.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

The term “promoter” as used herein is defined as a DNA sequence recognized by the synthetic machinery of the cell, or introduced synthetic machinery, required to initiate the specific transcription of a polynucleotide sequence.

As used herein, the term “promoter/regulatory sequence” means a nucleic acid sequence which is required for expression of a gene product operably linked to the promoter/regulatory sequence. In some instances, this sequence may be the core promoter sequence and in other instances, this sequence may also include an enhancer sequence and other regulatory elements which are required for expression of the gene product. The promoter/regulatory sequence may, for example, be one which expresses the gene product in a tissue specific manner.

A “constitutive” promoter is a nucleotide sequence which, when operably linked with a polynucleotide which encodes or specifies a gene product, causes the gene product to be produced in a cell under most or all physiological conditions of the cell.

An “inducible” promoter is a nucleotide sequence which, when operably linked with a polynucleotide which encodes or specifies a gene product, causes the gene product to be produced in a cell substantially only when an inducer which corresponds to the promoter is present in the cell.

The term “SAM file”, as used herein, refers to a type of text file format that contains the alignment information of one or more nucleotide or protein sequences that are mapped against one or more reference sequences. These files can also contain unmapped sequences.

The term “BAM file”, as used herein, refers to a file containing alignment information of various nucleotide or protein sequences that are mapped against one or more reference sequences in a binary file format. BAM files are smaller and more efficient for software to work with than SAM files, saving time and reducing costs of computation and storage.

The term “subject” is intended to include living organisms in which an immune response can be elicited (e.g., mammals). A “subject” or “patient,” as used therein, may be a human or non-human mammal. Non-human mammals include, for example, livestock and pets, such as ovine, bovine, porcine, canine, feline and murine mammals. Preferably, the subject is human.

The phrase “under transcriptional control” or “operatively linked” as used herein means that the promoter is in the correct location and orientation in relation to a polynucleotide to control the initiation of transcription by RNA polymerase and expression of the polynucleotide.

A “vector” is a composition of matter which comprises an isolated nucleic acid and which can be used to deliver the isolated nucleic acid to the interior of a cell. Numerous vectors are known in the art including, but not limited to, linear polynucleotides, polynucleotides associated with ionic or amphiphilic compounds, plasmids, and viruses. Thus, the term “vector” includes an autonomously replicating plasmid or a virus. The term should also be construed to include non-plasmid and non-viral compounds which facilitate transfer of nucleic acid into cells, such as, for example, polylysine compounds, liposomes, and the like. Examples of viral vectors include, but are not limited to, Sendai viral vectors, adenoviral vectors, adeno-associated virus vectors, retroviral vectors, lentiviral vectors, and the like.

Ranges: throughout this disclosure, various embodiments of the present disclosure can be presented in a range format. It should be understood that the description in range format is merely for convenience and brevity and should not be construed as an inflexible limitation on the scope of the present disclosure. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The methods as described herein involves a comprehensive screening of mutational effects on gene activity. In some embodiments, a gene activity can be used interchangeably with protein activity. In some embodiments, the mutational effects of the gene or protein activity is utilized to generate a profile.

Further, the method as provided herein includes the generation of a gene mutation library of cDNAs of a unique variant. As used herein, the unique variant comprises one or more amino acid substitution in a wildtype protein. In some embodiments, the unique variant can comprise a single amino acid substitution. In other cases, the unique variants can comprise at least two amino acid substitutions. In some embodiments, each unique variant is compared to the wildtype target protein. In some cases, the comparison is based on a statistical model to test a specific hypothesis regarding the biological activity of each unique variant. In some embodiments, the specific hypothesis regarding the biological activity of each unique variant can include but is not limited to loss-of-function, gain-of-function, substantially similar activity to the wildtype target protein, a drug resistant, or a drug susceptible. In some embodiments, the specific hypothesis regarding the biological activity of each unique variant can comprise a loss-of-function variant, a gain-of-function variant, a unique variant with substantially similar activity to the wildtype target protein, a drug resistant variant, or a drug susceptible variant. In some embodiments, a unique variant is a loss-of-function variant, a gain-of-function variant, a variant with substantially similar activity to the wildtype protein, a drug resistant variant, or a drug susceptible variant. In some embodiments, each cDNA comprises a single unique variant. In some embodiments, a cDNA library is generated where each cDNA in the library encodes a unique variant comprising an amino acid substitution in a wildtype target protein. Moreover, the cDNA library can comprise cDNAs encoding unique variant(s) each containing an amino acid substitution in a different amino acid in the wildtype protein. In some embodiments, the cDNA library can comprise mutations in each and every residue in the wildtype protein. In some instances, a cDNA library is generated comprising cDNAs each containing at least one amino acid substitution in a target protein. In some embodiments, the cDNAs results in the expression of the target protein comprising a mutation at one or more amino acid position(s).

The unique proteins as disclosed herein can comprise a wildtype protein comprising one or more amino acid substitutions. In some embodiments, the wildtype protein can include, but is not limited to, a blood coagulation protein such as Factor IX (FIX), Factor VIII (FVIII), Factor VIIa (FVIIa), Von Willebrand Factor (VWF), Factor FV (FV), Factor X (FX), Factor XI (FXI), Factor XII (FXII), thrombin (FII), protein C, protein S, tPA, PA-1, tissue factor (TF), ADAMTS 13 protease or fragments thereof. In other embodiments, the wildtype protein includes, but is not limited to, mmunoglobulins, cytokines such IL-1 alpha, IL-1 beta, IL-2, IL-3, IL-4, IL-5, IL-6, IL-11, colony stimulating factor-1 (CSF-1), M-CSF, SCF, GM-CSF, granulocyte colony stimulating factor (G-CSF), EPO, interferon-alpha (IFN-alpha), consensus interferon, IFN-beta, IFN-gamma, IFN-omega, IL-7, IL-8, IL-9, IL-10, IL-12, IL-13, IL-14, IL-15, IL-16, IL-17, IL-18, IL-19, IL-20, IL-21, IL-22, IL-23, IL-24, IL-31, IL-32 alpha, IL-33, thrombopoietin (TPO), angiopoietins, for example Ang-1, Ang-2, Ang-4, Ang-Y, the human angiopoietin-like polypeptides ANGPTL1 through 7, vitronectin, vascular endothelial growth factor (VEGF), angiogenin, activin A, activin B, activin C, bone morphogenic protein-1, bone morphogenic protein-2, bone morphogenic protein-3, bone morphogenic protein-4, bone morphogenic protein-5, bone morphogenic protein-6, bone morphogenic protein-7, bone morphogenic protein-8, bone morphogenic protein-9, bone morphogenic protein-10, bone morphogenic protein-11, bone morphogenic protein-12, bone morphogenic protein-13, bone morphogenic protein-14, bone morphogenic protein-15, bone morphogenic protein receptor IA, bone morphogenic protein receptor IB, bone morphogenic protein receptor II, brain derived neurotrophic factor, cardiotrophin-1, ciliary neutrophic factor, ciliary neutrophic factor receptor, cripto, cryptic, cytokine-induced neutrophil chemotactic factor 1, cytokine-induced neutrophil, chemotactic factor 2a, cytokine-induced neutrophil chemotactic factor 2B, β endothelial cell growth factor, endothelin 1, epidermal growth factor, epigen, epiregulin, epithelial-derived neutrophil attractant, fibroblast growth factor 4, fibroblast growth factor 5, fibroblast growth factor 6, fibroblast growth factor 7, fibroblast growth factor 8, fibroblast growth factor 8b, fibroblast growth factor 8c, fibroblast growth factor 9, fibroblast growth factor 10, fibroblast growth factor 11, fibroblast growth factor 12, fibroblast growth factor 13, fibroblast growth factor 16, fibroblast growth factor 17, fibroblast growth factor 19, fibroblast growth factor 2B, fibroblast growth factor 21, fibroblast growth factor acidic, fibroblast growth factor basic, glial cell line-derived neutrophic factor receptor α1, glial cell line-derived neutrophic factor receptor α2, growth related protein, growth related protein α, growth related protein β, growth related protein Y, heparin binding epidermal growth factor, hepatocyte growth factor, hepatocyte growth factor receptor, hepatoma-derived growth factor, insulin-like growth factor I, insulin-like growth factor receptor, insulin-like growth factor II, insulin-like growth factor binding protein, keratinocyte growth factor, leukemia inhibitory factor, leukemia inhibitory factor receptor α, nerve growth factor nerve growth factor receptor, neuropoietin, neurotrophin-3, neurotrophin-4, oncostatin M (OSM), placenta growth factor, placenta growth factor 2, platelet-derived endothelial cell growth factor, platelet derived growth factor, platelet derived growth factor A chain, platelet derived growth factor AA, platelet derived growth factor AB, platelet derived growth factor B chain, platelet derived growth factor BB, platelet derived growth factor receptor α, platelet derived growth factor receptor β, pre-B cell growth stimulating factor, stem cell factor (SCF), stem cell factor receptor, TNF, including TNF0, TNF1, TNF2, transforming growth factor α, transforming growth factor β, transforming growth factor β1, transforming growth factor β1.2, transforming growth factor β2, transforming growth factor β3, transforming growth factor β5, latent transforming growth factor β1, transforming growth factor β binding protein I, transforming growth factor β binding protein II, transforming growth factor (binding protein III, thymic stromal lymphopoietin (TSLP), tumor necrosis factor receptor type I, tumor necrosis factor receptor type II, urokinase-type plasminogen activator receptor, vascular endothelial growth factor, or active fragments thereof. Other non-limiting examples of the wildtype protein includes alpha-, beta-, and gamma-interferons, colony stimulating factors including granulocyte colony stimulating factors, fibroblast growth factors, platelet derived growth factors, phospholipase-activating protein (PUP), insulin, plant proteins such as lectins and ricins, tumor necrosis factors and related alleles, soluble forms of tumor necrosis factor receptors, interleukin receptors and soluble forms of interleukin receptors, growth factors such as tissue growth factors, such as TGFαs or TGFβs and epidermal growth factors, hormones, somatomedins, pigmentary hormones, hypothalamic releasing factors, antidiuretic hormones, prolactin, chorionic gonadotropin, follicle-stimulating hormone, thyroid-stimulating hormone, tissue plasminogen activator, and immunoglobulins such as IgG, IgE, IgM, IgA, and IgD, a galactosidase, α-galactosidase, β-galactosidase, DNAse, fetuin, leutinizing hormone, estrogen, corticosteroids, insulin, albumin, lipoproteins, fetoprotein, transferrin, thrombopoietin, urokinase, DNase, integrins, thrombin, hematopoietic growth actors, leptin, glycosidases, and fragments thereof, or any fusion proteins comprising any of the above mentioned proteins or fragments thereof. In some embodiments, the wildtype protein is a protein that binds Erbb2, Erbb2 variants, or fragments thereof. In some cases, the wildtype protein is not a protein that binds Erbb2, Erbb2 variants, or fragments thereof.

Also provided herein are engineered human or mammalian cell lines that encode a receptor, optionally a surface-expressed reporter, operably linked to a reporter system. In some cases, the receptor and the reporter system can indicate if one of the unique variant(s) binds to and modulates a biological activity of the receptor. In some embodiments, the receptor is a surface-expressed receptor. The term “surface-expressed receptor” refers to cell surface receptors (membrane receptors, transmembrane receptors) that are embedded in the plasma membrane of cells. For example, the surface-expressed receptor can include cytokine receptor, chemokine receptor, interferon receptor, 5T4, A33, activin receptor, adrenomedullin receptors, AFP, AGS-5, ALK, annexin, AXL, B7-H3, B7-H4, BAGE proteins, BCMA, Bombesin, C33 antigen, C4.4a, C-type lectin-like (-receptor), CA19.9, CA-125, CADMI, CAIX, CanAg, CAR, carbonic anhydrase, Caveolin-1, CCK2R, CD4, CD10, CD19, CD20, CD21, CD22, CD25, CD27, CD30, CD33, CD37, CD38, CD44, CD51, CD57, CD70, CD73, CD74, CD79a, CD79b, CD80, CEA, CEACAMs, c-kit, claudin, chemokine receptors (i.e., CXCR4, CXCRS), c-Met, Cripto-1, DEC-205, Derlin-1, Desmoglein-3, Dlk-1, DLL3, DS6, E-cadherin, E-Selectin, EAG-1, ED-B, EpCAM, EGFR, EGFRVIII, emmprin, endothelin receptor, ErbB2/Her2, ErbB3, ErbB4, ETV6-AML, Ephrin type-A receptor, Epiregulin, ETA, FAP-alpha, FcyR's, FGFR, FOLR1, Frizzled, Fyn3, Galectin, Ganglioside, GCC, GD2, GD3, GloboH, lypican-3, GLUTS, GPNMB, G-protein coupled receptors (i.e., GPR49), gp100, Hsp, HLA/B-raf, HLA-DR, HLA/k-ras, HLA MAGE-A3, HMW-MAA, hTERT, ICAM-3, IGF-R, IL-13-R, LICAM, laminin receptor, LIV1, LMP2, LRP5, LRP6, MAGE proteins, MART-1, melanotransferrin, mesothelin, metalloproteinase, ML-IAP, Mucins, Mud, Mud 6 (CA-125), MU M1, N-cadherin, NA17, NCAM-1, Nectin4, Notch, NP-55, NRP1, NY-BR1, NY-BR62, NY-BR85, NY-ES01, PLAC1, PRLR, PRAME, prominin-1, PSMA (FOLH 1), RON, SLC44A4, SLITRK6, Steap-1, Steap-2, surviving, syndecan, TAG-72, TF, TGF-p, TMPRSS2, TMEFF2, TNFR, Tn, TROP2, TRP-1, TRP-2, TWEAKR, tyrosinase, uroplakin-3 and VEGFR.

As used herein, the term “library,” refers to a pool of at least two polynucleotides, cell clones, molecules, or proteins. In certain embodiments, a library is used to screen for cDNAs encoding unique variants. In some embodiments, the library is a plasmid cDNA library. In other embodiments, the library is a viral library. The viral library, for example, can comprise a lentiviral library, an adenoviral library, or a retroviral library. In other embodiments, a plasmid cDNA library of cDNA is converted into a viral library. In other embodiments, a library of drugs is mixed with a single cell clone, and then a biological assay is used to discern which drugs cause a response in the cell clone. A plasmid cDNA library can comprise 100, 1,000, 10,000, 100,000, or 1 million different cDNA molecules. In other embodiments, a plasmid cDNA library comprises 100, 1,000, 10,000, 100,000, or 1 million cDNAs further comprising a unique molecular identifier (UMI) or a barcode group, or cells engineered to express the cDNA with UMIs or barcodes as RNA.

In some embodiments, each cDNA of the cDNA library is incorporated into one plasmid, such that each cDNA of the cDNA library is individually and independently incorporated into its own plasmid, forming a plasmid cDNA library. In some embodiments, each plasmid of the plasmid cDNA library has only one unique cDNA of the plurality of cDNAs, and wherein each plasmid in the plasmid cDNA library further comprises a unique molecular identifier (UMI) or a barcode group. In some embodiments, the cDNA library is packaged into a viral vector, thereby generating a viral library. In some embodiments, the viral library comprises virions. In some embodiments, each virion comprises only one viral plasmid of the cDNA library. In some embodiments, the viral library is a retroviral library, an adenoviral library, or a lentiviral library. In some embodiments, the viral library is a lentiviral library. In some embodiments, the lentiviral vector includes an expression cassette. In some embodiments, the expression cassette can include a promoter operably linked to the polynucleotide sequence encoding a unique variant. In some embodiments, the promoter operably linked to the polynucleotide encoding the unique variant is inducible. In some embodiments, the target cells are transduced with the cDNA library lentiviral pool at a low multiplicity of infection (MOI) such that >90% of the target cells express a single unique variant. In other embodiments, the target cells are transduced with the lentiviral library lentiviral pool at a high multiplicity of infection so that target cells express unique variants. The terms “multiplicity of infection” or “MOI” are used according to its plain ordinary meaning in Virology and refers to the ratio of infectious agent (e.g., a virus) to the target (e.g., a cell) in a given area or volume. In embodiments, the area or volume is assumed to be homogenous. In some embodiments, the MOI is at least 0.001, at least 0.01, at least 0.01, at least 0.1, at least 0.2, at least 0.3, at least 0.4, at least 0.5, at least 0.6, at least 0.7, at least 0.8, at least 0.9, or at least 1.

In some embodiments presented herein is a method of preparing a plurality of a plasmid cDNA library from a plurality of cDNAs, the method comprising incorporating a tag into the cDNA to provide a plurality of tagged cDNA samples, wherein the cDNA in each tagged cDNA sample encodes a unique variant. In one embodiment, the tag comprises a cell-specific identifier sequence and a unique molecular identifier (UMI) sequence. In some embodiments, the tag comprises a cell-specific identifier sequence without the UMI. In some embodiments, the tag comprises a barcode. The method further comprises pooling the tagged cDNA samples;

optionally amplifying the pooled cDNA samples to generate a cDNA library comprising double-stranded cDNA, thereby generating a plurality of tagged cDNA. In some embodiments, the cDNA of the plurality of cDNAs further comprises a UMI or a barcode group. In some embodiments, the UMI or the barcode groups can be immobilized to a solid support. For example, the solid support can be one or more beads. Thus, in certain embodiments, a plurality of beads can be presented, wherein each bead in the plurality bears a unique sample barcode and/or UMI sequence. In some embodiments, each cDNA of the cDNA library are contacted with one or more beads having a unique set of sample barcodes and/or UMI sequences in order to identify the cDNA of the cDNA library. In some embodiments, purified nucleic acid from a transduced cell are contacted with one or more beads having a unique set of sample barcodes and/or UMI sequences in order to identify the purified cDNA from the transduced cell.

As used herein, the term “transfected” or “transformed” or “transduced” refers to a process by which exogenous nucleic acid is transferred or introduced into the host cell. A “transfected” or “transformed” or “transduced” cell is one which has been transfected, transformed or transduced with a cDNA encoding a unique variant. The cell includes the primary subject cell and its progeny. In some embodiments, a library of transduced cells can comprise about 100, about 1,000, about 10,000, about 100,000, about 500,000, about 1 million, about 5 million, or about 10 million transduced cells. In some embodiments, the library of transduced cells can comprise from about 500,000 cells to about 10 million cells. In some embodiments, the library of transduced cells comprises about 10,000 cells, 20,000 cells, 30,000 cells, 40,000 cells, 50,000 cells, 60,000 cells, 70,000 cells, 80,000 cells, 90,000 cells, 100,000 cells, 150,000 cells, 200,000 cells, 300,000 cells, 400,000 cells, 500,000 cells, 600,000 cells, 700,000 cells, 800,000 cells, 900,000 cells, 1,000,000 cells, 2,000,000 cells, 3,000,000 cells, 4,000,000 cells, 5,000,000 cells, 6,000,000 cells, 7,000,000 cells, 8,000,000 cells, 9,000,000 cells or 10,000,000 cells. In some embodiments, the cells in the library of transduced cells are isolated and purified. In some embodiments, a library of isolated and purified transduced cells comprising from about 10,000 transduced cells to about 10 million cells, wherein each isolated and purified transduced cell contains a plasmid comprising a cDNA that encodes for a unique variant of a wildtype protein, such that the transduced cell further comprises a cell-surface receptor operably coupled to a reporter system, and wherein the cell-surface receptor is capable of interrogating each unique variant. In some cases, each isolated and purified transduced cell further comprises on its surface a unique variant.

In some cases, a high throughput, and optionally computerized or robot implemented, system for identifying protein is provided. In such embodiments, the method provided herein can comprise libraries of plasmid cDNAs and transduced cells arranged in a multiplicity of compartments. With respect to plasmids, the libraries contain compartments can contain a plasmid for expressing a cDNA encoding a unique variant as disclosed herein. Such libraries can be very efficiently used to transduce cells to produce a library of cells in a multiplicity of compartments, each of which contains cells transduced with one vector. The libraries can optionally be propagated in packaging cells prior to their use in cell transduction.

The libraries of transduced cells can be analyzed for the effects of the unique variants by use of machine implemented microarray or macro-array technologies. For example, a machine implemented technology can be used to determine a large number of sequences via a single “chip” used for the hybridization of mRNA encoding the unique variants, or the corresponding cDNA, isolated from cells. The libraries of transduced cells may also be subject to further treatment or changing conditions before analysis of effects on cellular factors. The cells, and hence effects on cellular factors, may also be analyzed temporally. The function of a gene or a protein can also be assessed through cellular differentiation and function in vivo in culture, or after transplantation in an animal model, or in human or non-human primates.

Further, in some embodiments, the cells used in the cell-based assay can comprise a nucleic acid encoding a reporter molecule (e.g., a reporter protein) is operably linked to a promoter and/or enhancer responsive to activity of the unique variant. A “promoter” refers to a control region of a nucleic acid at which initiation and rate of transcription of the remainder of a nucleic acid sequence are controlled. A promoter drives transcription or of the nucleic acid sequence that it regulates, thus, it is typically located at or near the transcriptional start site of a gene. A promoter, in some embodiments, is 100 to 1000 nucleotides in length. A promoter may also contain sub-regions at which regulatory proteins and other molecules may bind, such as RNA polymerase and other transcription factors. Promoters may be constitutive {e.g., CAG promoter, cytomegalovirus (CMV) promoter), inducible (also referred to as activatable), repressible, tissue-specific, developmental stage-specific or any combination of two or more of the foregoing. A promoter is considered to be “operably linked” when it is in a correct functional location and orientation relative to a sequence of nucleic acid that it regulates {e.g., to control (“drive”) transcriptional initiation and/or expression of that sequence). A promoter, in some embodiments, is naturally associated with a nucleic acid and may be obtained by isolating the 5′ non-coding sequence(s) located upstream of the coding region of the given nucleic acid. Such a promoter is referred to as an “endogenous” promoter.

In some embodiments, a promoter can be a constitutively active promoter (i.e., a promoter that is constitutively in an active/“ON” state), it may be an inducible promoter (i.e., a promoter whose state, active/“ON” or inactive/“OFF”, is controlled by an external stimulus, e.g., the presence of a particular temperature, compound, or protein), it may be a spatially restricted promoter (i.e., transcriptional control element, enhancer, etc.) (e.g., tissue specific promoter, cell type specific promoter, etc.), and it may be a temporally restricted promoter. Suitable promoters can be derived from viruses and can therefore be referred to as viral promoters, or they can be derived from any organism, including prokaryotic or eukaryotic organisms. Suitable promoters can be used to drive expression by any RNA polymerase (e.g., pol I, pol II, pol III). Exemplary promoters include, but are not limited to the SV40 early promoter, mouse mammary tumor virus long terminal repeat (LTR) promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, a human U6 small nuclear promoter (U6), a human H1 promoter (H1), and the like. Examples of inducible promoters include, but are not limited to T7 RNA polymerase promoter, T3 RNA polymerase promoter. Isopropyl-beta-D-thiogalactopyranoside (IPTG)-regulated promoter, lactose induced promoter, heat shock promoter, Tetracycline-regulated promoter (e.g., Tet-ON, Tet-OFF, etc.), Steroid-regulated promoter, Metal-regulated promoter, estrogen receptor-regulated promoter, etc. Inducible promoters can therefore be regulated by molecules including, but not limited to, doxycycline; RNA polymerase, e.g., T7 RNA polymerase; an estrogen receptor; an estrogen receptor fusion; etc. In some embodiments, the promoter is a doxycycline inducible promoter.

In some embodiments, the promoter is a spatially restricted promoter (i.e., cell type specific promoter, tissue specific promoter, etc.) such that in a multi-cellular organism, the promoter is active (i.e., “ON”) in a subset of specific cells. Spatially restricted promoters may also be referred to as enhancers, transcriptional control elements, control sequences, etc. Any convenient spatially restricted promoter may be used and the choice of suitable promoter (e.g., a brain specific promoter, a promoter that drives expression in a subset of neurons, a promoter that drives expression in the germline, a promoter that drives expression in the lungs, a promoter that drives expression in muscles, a promoter that drives expression in islet cells of the pancreas, etc.) will depend on the organism.

In some embodiments, the promoter and/or enhancer responsive to activity of the unique variant is an expression control sequence. Expression and cloning vectors usually contain a promoter that is recognized by the host organism and is operably linked to the nucleic acid encoding the polypeptide (e.g., the reporter polypeptide). Suitably, the expression control sequences are eukaryotic promoter systems in vectors capable of transforming or transfecting eukaryotic host cells. Once the vector has been incorporated into the appropriate host, the host is maintained under conditions suitable for high level expression of the nucleotide sequences following T cell activation. In some embodiments, the method provides cells of human or mammalian cell lines comprising nucleic acid encoding a reporter molecule under the control of a promoter responsive to activation by the unique variant. In some embodiments, expression reporter vectors used in eukaryotic host cells (yeast, fungi, insect, plant, animal, human, or nucleated cells from other multicellular organisms) will also contain sequences necessary for the termination of transcription and for stabilizing the mRNA. Such sequences are commonly available from the 5′ and, occasionally 3′, untranslated regions of eukaryotic or viral DNAs or cDNAs. One useful transcription termination component is the bovine growth hormone polyadenylation region.

In some embodiments, the method provides vectors for the expression of the unique variants in cells of human or mammalian cell lines. Vector components generally include, but are not limited to, one or more of the following, a signal sequence, an origin of replication, one or more marker genes, a multiple cloning site containing recognition sequences for numerous restriction endonucleases, an enhancer element, a promoter (e.g., an enhancer element and/or promoter responsive to activation), and a transcription termination sequence. In some embodiments, the vector is a plasmid. In other embodiments, the vector is a recombinant viral genome; e.g., a recombinant lentiviral genome, a recombinant retrovirus genome, a recombinant adeno-associated viral genome. The vectors containing the cDNA library be transduced into a cell of human or mammalian cell lines.

In some embodiments, the method provides a cell-based assay to detect the activity of the unique variants by contacting a population of cells comprising a reporter assay system responsive to the unique variants. In some embodiments, the cells are mammalian cells. Mammalian cells can include but is not limited to, mouse, rat, hamster or human cells. In some embodiments the cells are human cells. In some embodiments, the cells exhibit increase biological activity. In some embodiments, the population of cells is a population of immortalized cells (e.g., an immortalized cell line).

In some embodiments, cells in which the cDNA libraries have been introduced are screened for activation of a receptor. For example, stable clones can be isolated by limiting dilution and screened for their binding response to a receptor. In some embodiments, stable reporter T cells are screened with more than about any of 1 μg/mL, 2 μg/mL, 3 μg/mL, 4 μg/mL, 5 μg/mL, 6 μg/mL, 7 μg/mL, 8 μg/mL, 9 μg/mL, or 10 μg/mL receptor. In some embodiments, the receptor is a surface-based receptor, a costimulatory molecule, an accessory molecule, an immune checkpoint molecule, a member of the TNF family or the TNF family receptor, a cytokine receptor, a chemokine receptor, or an adhesion molecule.

Provided herein is a method for screening large numbers of compounds for activity with respect to a particular biological function requires preparing arrays of cells for parallel handling of cells and reagents. Standard 96 well microtiter plates which are 86 mm by 129 mm, with 6 mm diameter wells on a 9 mm pitch, are used for compatibility with current automated loading and robotic handling systems. The microplate is typically 20 mm by 30 mm, with cell locations that are 100-200 microns in dimension on a pitch of about 500 microns. Microplates may consist of coplanar layers of materials to which cells adhere, patterned with materials to which cells will not adhere, or etched 3-dimensional surfaces of similarly pattered materials. For the purpose of the following discussion, the terms ‘well’ and ‘microwell’ refer to a location in an array of any construction to which cells adhere and within which the cells are imaged. Microplates may also include fluid delivery channels in the spaces between the wells. The smaller format of a microplate increases the overall efficiency of the system by minimizing the quantities of the reagents, storage and handling during preparation and the overall movement required for the scanning operation. In addition, the whole area of the microplate can be imaged more efficiently, allowing a second mode of operation for the microplate reader as described later in this document.

Provided herein is a method that comprises assessing a unique variant's biological activity using fluorescent and luminescent reagents to measure the temporal and spatial distribution, content, and activity of intracellular ions, metabolites, macromolecules, and organelles. Classes of these reagents include labeling reagents that measure the distribution and number of molecules in living and fixed cells, environmental indicators to report signal transduction events in time and space, and fluorescent protein biosensors to measure target molecular activities within living cells.

The method of the present disclosure is based on the high affinity of fluorescent or luminescent molecules for specific cellular components. The affinity for specific components is governed by physical forces such as ionic interactions, covalent bonding (which includes chimeric fusion with protein-based chromophores, fluorophores, and lumiphores), as well as hydrophobic interactions, electrical potential, and, in some cases, simple entrapment within a cellular component. The luminescent probes can be small molecules, labeled macromolecules, or genetically engineered proteins, including, but not limited to green fluorescent protein chimeras.

As used herein the method as described includes a reporter assay. A “reporter assay” as used herein, refers to an analytical method that enables the biological characterization of a stimulus by monitoring the induction of expression of a reporter in a cell. The stimulus leads to the induction of intracellular signaling pathways that result in a cellular response that typically includes modulation of gene transcription.

In some examples, stimulation of cellular signaling pathways result in the modulation of gene expression via the regulation and recruitment of transcription factors to upstream non-coding regions of DNA that are required for initiation of RNA transcription leading to protein production. Control of gene transcription and translation in response to a stimulus is required to elicit the majority of biological responses such as cellular proliferation, differentiation, survival and immune responses. These non-coding regions of DNA, also called enhancers, contain specific sequences that are the recognition elements for transcription factors which regulate the efficiency of gene transcription and thus, the amount and type of proteins generated by the cell in response to a stimulus. In a reporter assay, an enhancer element and minimal promoter that is responsive to a stimulus is engineered to drive the expression of a reporter gene using standard molecular biology methods. The DNA is then transfected into a cell, which contains all the machinery to specifically respond to the stimulus, and the level of reporter gene transcription, translation, or activity is measured as a surrogate measure of the biological response.

In some embodiments, the method as disclosed comprise a cell-based assay where a receptor is operably linked to a reporter system. In some embodiments, the receptor is a surface-expressed receptor. In some embodiments, a reporter system comprises a reporter molecule. A reporter molecule may be any molecule for which an assay can be developed to measure the amount of that molecule that is produced by the cell in response to the stimulus. For example, a reporter molecule may be a reporter protein that is encoded by a reporter gene that is responsive to the stimulus. Commonly used examples of reporter molecules include but are not limited to luminescent proteins such as luciferase, which emit light as a by-product of the catalysis of substrate which can be measured experimentally. Luciferases are a class of luminescent proteins that are derived from many sources including firefly luciferase (from the species, Photinus pyralis), Renilla luciferase from sea pansy (Renilla reniformis), click beetle luciferase (from Pyrearinus termitilluminans), marine copepod Gaussia luciferase (from Gaussia princeps), and deep sea shrimp Nano luciferase (from Oplophorus gracilirostris). Firefly luciferase catalyzes the oxygenation of luciferin to oxyluciferin resulting in the emission of a photon of light while other luciferases such as Renilla emit light by catalyzing coelenterazine. The wavelength of light emitted by different luciferase forms and variants can be read using different filter systems, which facilitate multiplexing. The amount of luminescence is proportional to the amount of luciferase expressed in the cell and luciferase genes have been used as a sensitive reporter to evaluate the impact of a stimulus to elicit a biological response.

In some embodiments, the method provides cell-based assays to detect the unique variants as disclosed herein where the cells of a human or a mammalian cell line encodes a reporter construct that is responsive to the unique variant biological activation. In some embodiments, the reporter construct comprises a luciferase. In some embodiments, the luciferase is a firefly luciferase (e.g., from the species Photinus pyralis), Renilla luciferase from sea pansy (e.g., from the species Renilla reniformis), click beetle luciferase (e.g., from the species Pyrearinus termitilluminans), marine copepod Gaussia luciferase (e.g., from the species Gaussia princeps), and deep sea shrimp Nano luciferase (e.g., from the species Oplophorus gracilirostris). In some embodiments, expression of luciferase in the cells of the human or mammalian cell line indicates the binding activity between the unique variant and the reporter protein. In some instances, the reporter construct encodes a β-glucuronidase (GUS); a fluorescent protein such as Green fluorescent protein (GFP), red fluorescent protein (RFP), blue fluorescent protein (BFP), yellow fluorescent protein (YFP) and variants thereof; a chloramphenicoal acetyltransferase (CAT); a β-galactosidase; a β-lactamase; or a secreted alkaline phosphatase (SEAP).

In some embodiments, the method further provides a sequencing method to assess the mutational effect on gene or protein activity. In some embodiments, the sequencing method is next-generation sequencing, sanger sequencing, whole genome sequencing, RNA sequencing or shot gun sequencing. In some embodiments, the sequencing method is next-generation sequencing. In some cases, the next-generation sequencing results in a sequence dataset. The sequence data can comprise a read depth for each unique variant of about 2000× to about 90,000× sequencing coverage. In some embodiments, the read dept for each unique variant is at least about 1000 times, 2000 times, 3000 times, 4000 times, 5000 times, 6000 times, 7000 times, 8000 times, 9000 times, 10000 times, 50,000 times, or 100,000 times. It is performed for several sequencing reactions. Less than 50,000 or less than 100,000 sequencing reactions may be performed. An exemplary read depth is about 1000 to about 50,000, or about 2000 to about 90,000, or about 5000 to about 100,000 reads per locus (base position). In some embodiments, the method further comprises validating the method by comparing the biological activity of a subset of unique variants analyzed by the method to a previously determined activity of a wildtype target protein. In other embodiments, the method further comprises validating the method by comparing true negatives as determined by the method to true negatives determined by an independent method. In some cases, the method further comprises comparing the biological activity of a subset of unique variants analyzed by the method to independent testing of a set of separate clones comprising the unique variant(s). In some embodiments, the method is further validated by comparing method results among one or more different samples. In some cases, the method is further validated by comparing results in two different cell lines.

Also provided herein, is a method comprising generating an oligonucleotide sequence dataset. In some embodiments, the method further comprises analyzing or analysis of the oligonucleotide sequence dataset using a computer. In some instances, the analysis or analyzing includes (a) generating a unique variant-UMI index library using long reads from next-generation sequencing (NGS); (b) identifying UMI counts and barcode bins by analyzing short reads of flow-sorted groups; (c) calculating an activity score from a UMI-barcode read counts comprising the UMI counts and barcode bins of (b); (d) assessing an effect of the unique variant(s) on biological activity by calculating read percent of the reporter protein, such as a fluorescent protein, and p-value; and (e) accurately and quantitatively assessing activities of the unique variant(s) with respect to previously characterized wildtype. In some embodiments, the fluorescent protein is GFP, and wherein biological activity comprises a gene activity or a protein activity.

### EXAMPLES

### Example 1: Generation and Analysis of a Gene Mutation Library (GML)

To produce a comprehensive mutation effect on gene activity (MEGA)-mutation activity profile (Map), a GML is analyzed with the assay system described.

### Creating a Gene Mutation Library (GML)

A library of mutant cDNA molecules for a target gene was designed. This library covers a promoter, coding region, 3′UTR a mini gene containing and intron with splice sites or a portion of any of the above.

### Generation of UMI-Barcoded Variant Plasmid Libraries.

A doxycycline-inducible lentiviral plasmid was made by PCR amplifying the TreTIGHT doxycycline-inducible promoter from pSSI9343 (a kind gift from Sierra Sciences, Reno, NV) using oHI-00176 (with a 5′ overhang complementary to the sequence upstream of SnaBI restriction site in pLJM1_MCS) and oHI-00177 (with a 5′ overhang complementary to the sequence downstream of BmtI in pLJM1_MCS) and gel purified using Nucleospin Gel and PCR Cleanup Kit (Macherey-Nagel, Allentown, PA). The PCR product was then mixed with SnaBI-BmtI linearized pLJM1_MCS (2:1 insert: vector molar ratio) and combined in a HiFi DNA Assembly reaction containing NEBuilder HiFi DNA Assembly Master Mix (New England Biolabs, Ipswich, PA. The reaction was incubated for 15 min at 50° C. to create a final lentiviral plasmid with a multiple cloning site downstream of the doxycycline-inducible TreTIGHT promoter.

A double-stranded (ds) DNA library containing codon optimized full-length Erbb2 cDNAs with sequences for all the possible single amino acid mutants in the juxtamembrane domain and tyrosine kinase domain were synthesized by Twist Bioscience (San Francisco, CA). The ds-DNAs from each well of the 96-well plates were pooled and cDNAs were purified using the Nucleospin Gel and PCR Cleanup Kit (Macherey-Nagel, Allentown, PA). The synthesized cDNAs contain a 5′ overhang sequence upstream of the Kozak-containing the Erbb2 ATG start codon that is complementary to the sequence upstream of the BmtI site (BmtI site in lowercase; (5′-GGTTTAGTGAACCGTCAGATCCgctagc-3′) in lentiviral plasmid pHI-00104 and 3′ overhang sequence (5′-TCGATCCCGTACCGAGGAGATCTG-3′) downstream of the HER2 stop codon that is complementary to the 3′ end of an oligo (oHI-00165) containing 32 nucleotides of randomized DNA sequence. The randomized DNA sequence in oHI-00165 will be cloned into the 3′ untranslated region (UTR) of the final plasmid library such that every cDNA molecule is barcoded with a Unique Molecular Identifier (UMI).

The 5′ end of oHI-00165 contains an overhang sequence (MluI site in lower case; 5′-ATTTGTCTCGAGGTCGATTCGAATacgcgt-3′) complementary to the sequence downstream of the MluI site in pHI-00104. The purified ds-DNA mutant library fragments, UMI oligo oHI-00165, and BmtI-MluI digested pHI-00104 lentiviral plasmid backbone were mixed in a 2:5:1 molar ratio of insert:oligo:vector and used in a 5×-scaled up HiFi DNA Assembly reaction with NEBuilder HiFi DNA Assembly Master Mix (New England Biolabs, Ipswich, PA) and incubated for 1 h at 50° C.. The assembly reaction was cleaned up using Monarch PCR & DNA Cleanup Kit (New England Biolabs, Ipswich, PA) and drop dialyzed on MF-Millipore Membrane filter, 0.025 μm pore size (Millipore Sigma, #VSWPO2500, Burlington, MA).

The purified and dialyzed assembly reaction mixture was electroporated into Endura electrocompetent cells (Lucigen, Middleton, WI), plated on pre-warmed LB ampicillin plates, and incubated for 16 hours at 37° C. Transformants were scraped and the plasmid library from the pooled cell suspension was isolated using the EndoFree Plasmid Mega kit (Qiagen, #12381, Germantown, MD). PacBio long-read sequencing was performed at the DNA Sequencing Center, Brigham Young University, Salt Lake City, UT to sequence the full-length Erbb2 cDNA and UMI regions of the GML. PCR (15 cycles) was used to generate a sequence data set containing DNA fragments for long-read sequencing using Q5 High Fidelity DNA Polymerase (New England Biolabs, Ipswich, PA) with PCR primers oHI-20-00030 (targeted to the 5′ untranslated region upstream of the ATG start codon in the Erbb2 gene) and oHI-00203 (targeted to the 3′ untranslated region downstream of the UMI site). Another non-PCR based method was also used for long-read sequencing by gel-purifying the DNA fragment from digesting the gene mutation library with NheI-Mlul restriction enzymes.

### Construction of Individual Erbb2 Variant Alleles for Controls.

Plasmid pcDNA3.1+/C-(K)-DYK-Erbb2 containing full-length wild-type Erbb2 and PCR amplified using Q5 High Fidelity DNA Polymerase (New England Biolabs, Ipswich, PA) with PCR primers (with a 5′ overhang containing NheI/BmtI restriction site and Kozak site upstream of the ATG start codon) and oHI-20-00009 (with a 5′ overhang containing a SalI restriction site downstream of a stop codon to replace the C-terminal DYK tag). The PCR product was digested with DpnI and A-tails were added by incubating with Taq DNA polymerase (New England Biolabs, Ipswich, PA) for 20 min at 72° C.. The PCR product was TOPO cloned into pCR4-TOPO vector using the TOPO TA Cloning kit (Thermo Fisher Scientific, Hampton, NH) to create pHI-00062. Erbb2 was subcloned from pHI-00062 into BmtI-SalI sites of intermediate plasmid pHI-001 to create pHI-00065, and subcloned again from pHI-00065 into BmtI-AsiSI sites of lentiviral plasmid pLJM1 MCS to create pHI-00076 where Erbb2 is expressed by the CMV promoter.

The TreTIGHT doxycycline-inducible promoter was obtained via PCR amplification of pSSI9343 (a kind gift from Sierra Sciences in Reno, NV) using oHI-00176 (with a 5′ overhang complementary to the sequence upstream of SnaBI restriction site in pHI-00076) and oHI-00178 (with a 5′ overhang complementary to the sequence downstream of BmtI in pHI-00076) and gel-purified using Nucleospin Gel and PCR Cleanup Kit (Macherey-Nagel, Allentown, PA). The PCR product was then mixed as a 2:1 insert: vector molar ratio with SnaBI-BmtI linearized pHI-00076 in a HiFi DNA Assembly reaction with NEBuilder HiFi DNA Assembly Master Mix (New England Biolabs, Ipswich, PA) and incubated for 15 min at 50° C. This process produced a final lentiviral plasmid in which wild-type Errb2 mRNA is expressed by induction of the doxycycline-inducible TreTIGHT promoter.

To generate the YVMA Erbb2 GOF control mutant, pHI-00062 was PCR amplified using forward PCR primer oHI-20-00028 (flanking the AatII in HER2) and downstream PCR primer oHI-20-00029

(5′-CCCAGAAGGCGGGAGACATAgGGGGAGCCCACACCAGCCATCACGT
ATGCCATCACGTATGCTTCGTCTAAGATTTCTTTGTTGGCTTTGGGGGA
T-3′)

containing the 12-nt YVMA INDEL (underlined) and a silent mutation (lowercase font) to disrupt an NdeI site in Erbb2. The PCR product was gel-purified using Nucleospin Gel and PCR Cleanup Kit. The PCR product was then mixed as a 2:1 insert: vector molar ratio with an AatII-NdeI linearized pHI-00062 and mixed in a HiFi DNA Assembly reaction with NEBuilder HiFi DNA Assembly Master Mix (New England Biolabs, #E2621L, Ipswich, PA) and incubated for 15 min at 50° C. to create pHI-00067. The YVMA HER2 mutant was subcloned from pHI-00067 into BmtI-SalI sites of intermediate plasmid pHI-00060 to create pHI-00068 and subcloned again from pHI-00068 into BmtI-AsiSI sites of lentiviral plasmid pLJM1_MCS to create pHI-00077 where YVMA HER2 is expressed by the CMV promoter.

The TreTIGHT promoter was cloned into pHI-00077, similar to methods described above for wild-type Erbb2 to create a final lentiviral plasmid where YVMA HER2 is expressed by induction of the doxycycline-inducible TreTIGHT promoter. A codon-optimized Erbb2 was isolated as a clone from the GML, sequenced via Sanger Sequencing through the entire Erbb2 coding sequence and named as pHI-00163. The codon-optimized wild-type Erbb2 was PCR amplified with Q5 High Fidelity DNA Polymerase (New England Biolabs, Ipswich, PA) using PCR primers oHI-00414 and oHI-00415, TOPO cloned into pCR-Blunt-II-TOPO using the Zero Blunt TOPO PCR Cloning kit (Thermo Fisher Scientific; Hampton, NH) and sequenced verified via Sanger sequencing to create pHI-00196.

All other HER2 controls were made using QuikChange Lightning Site-Directed Mutagenesis Kit (Agilent, Santa Clara, CA) using a codon optimized wild-type Erbb2 as template. Primers used for mutagenesis were designed using Agilent's QuikChange Primer Design tool. Mutagenesis was carried out using a thermocycler with the PCR protocol: initial denaturation 95° C. for 2 min.; 18 cycles of denaturation at 95° C. for 2 min, annealing at 60° C. for 10 sec., extension at 68° C. for 3 min, 45 seconds; final extension 68° C. for 5 min; and ending with a hold of 4 degrees Celsius. Transformants were screened and Sanger sequenced through the mutagenesis site. Mutant Erbb2 cDNA was subcloned into NheI-SalI sites of plasmid pHI-00104 to create lentiviral plasmids where mutant Erbb2 controls are expressed using the doxycycline-inducible TreTIGHT promoter.

### Deep Sequencing of UMI-Barcoded Variant Libraries.

The variant library for the coding region was broken up into overlapping tiles with each tile having a single strand DNA of less than 300 bases to be synthesized as a single strand cDNAs. The collection of mutants in the library and each cDNA to be synthesized are designed with a custom program.

cDNAs in each pool were joined using the overlapping sequences. A HiFi DNA Assembly is a reaction used to join DNA fragments with overlapping sequences. During the assembly reaction, T5 exonuclease chews back the 5′ end of the DNA fragment leaving exposed overlapping sequences to allow complementary strands to anneal, HiFi DNA polymerase fills the gap by synthesizing DNA in 5′->3′ direction, and DNA ligase seals the nicks. This activity continues throughout the duration of the reaction leading to a highly efficient method of assembling DNA. Furthermore, this reaction can be carried out using a ssDNA oligo as a bridge between two exposed DNA ends if the oligo has 25-30 bp of overlapping DNA sequence with each end of the DNA fragments.

The capability of assembling ssDNA oligos into dsDNA fragments is the premise for using HiFi DNA Assembly for GML production. The ssDNA oligos can be in the form of synthesized oligo pools with oligo lengths ~200 nucleotides with thousands of different oligos within each pool. The oligo pools would be designed so that each oligo in the oligo pool would have 25 nucleotides of overlapping sequence on 5′ and 3′ ends that correspond to 25 bp on the 5′ and 3′ ends of the dsDNA fragment the oligos are being cloned into. This allows for 150 nucleotides (corresponding to a variant region of 50 amino acids) within the middle of the oligo to introduce desired mutations into a DNA sequence of interest corresponding protein coding sequences, promoter sequences, etc. In this approach there is no need for PCR amplification of the oligo pool. Preparation of the vector DNA for variant assembly involves inverse PCR to amplify the entire vector sequence excluding the variant region using PCR primers that provide the 25 bp of overlap corresponding to each oligo pool.

Furthermore, an entire coding sequence can be covered by dividing it into overlapping sections (or tiles) corresponding to the desired oligo lengths. Each tiled section would correspond to a separate oligo pool.

Once a variant assembly is complete for a GML, a separate UMI assembly reaction can be carried out on the purified variant assembly plasmid library. To do this, the plasmid library would be linearized by restriction enzymes and a UMI oligo with 25 nucleotides of overlapping sequence for assembly can be cloned into the UMI site (such as the 3′UTR).

### Producing a Lentiviral GML Library

Lenti-X 293T cells were plated into 2x 150 mm plates (12.5E6/plate) and allowed to attach for 24 h. Representation of ~40 cells per clone. Each plate was transfected with 10 μg of plasmid library (about 1.38E6 copies of plasmid DNA per clone), 9.5 μg psPAX2, 5.2 μg pMD2.G using Fugene HD as the transfection reagent at a 3 μL Fugene: 1 μg DNA ratio. Media was changed on plates 24 h post transfection. Viral supernatant was harvested about 72 h post-transfection, filtered through a 0.45 μM filter prior to being snap frozen in aliquots. Virus aliquots were thawed on ice and viral titer established using Lenti-X GOSTIX (Takara). Additional details are listed below:

### Counting and Seeding of Cells for Transfection

LentiX293T cells were grown in 100 mm petri dish in 10 ml complete DMEM media (DMEM+10% Fetal Calf Serum) and incubated CO2 incubator at 37° C. with a 5% CO2 atmosphere. Cells were trypsinized, and the cell pellet was resuspended in 3 ml of complete DMEM media. 3 million cells were seeded in a new petri dish having 10 ml complete DMEM media. The petri dish was labelled with the passage number and date of passage and the name of the person performing the culturing of the cells. The cells were cultured in CO₂ incubator at 37° C. at 5% CO2, until they reached 80-90% confluence. From the remaining cells, 4 million cells were seeded in 100 mm petri dish having 10 ml of complete DMEM media.

### Co-Transfection and Production of the Lentiviral Vectors

For each expression construct, 0.58E6 Lenti-X 293T cells were plated into 1x well of a 6-well plate and allowed to attach for 24 h. Each plate was transfected with 1.1 μg of plasmid library, 1.1 μg psPAX2, 0.6 μg pMD2.G using Fugene HD as the transfection reagent at a 3 μL Fugene: 1 μg DNA ratio. Media was changed on plates 24 h post transfection. Viral supernatant was harvested about 72 h post-transfection, filtered through a 0.45 μM filter prior to being snap frozen in aliquots. Virus aliquots were thawed on ice and viral titers established using Lenti-X GOSTIX (Takara). The cells were ready for transfection after 24 hours of seeding the 100 mm petri dish.

A transfection mix was prepared in 15 ml tube for each 100 mm petri dish containing: 8.5 μg pLjm1_Twist Tat Library; 7.6 μg pMDLG/pRRE: (plasmids were stored in the refrigerator); 4.0 μg pRSV/pRev; 4.0 μg pMD2.G. Sterile water was then added to bring the final volume to 613 μl. 87 μl 2 M CaCl₂) was also added. The plasmid, water and 2M CaCl₂) were then mixed, followed by adding 2×HBS 700 μl dropwise to the transfection mix above with gentle stirring of the 15 ml tube in a circular motion or slow vortex. The transfection mix was incubated for 15 minutes and then added dropwise to the 100 mm petri dish. Plates were incubated at 37 degrees Celsius for 8 hr (8-14 hours) overnight in a CO₂ incubator at 37° C. with a 5% CO₂ atmosphere. Calcium phosphate-containing medium was removed and replaced with 7 ml complete DMEM media (DMEM+10% FBS) and was incubated for 48 hours in a CO₂ incubator at 37° C. with a 5% CO₂ atmosphere. The spent media containing the lentivirus was collected from the completely confluent transfected LentiX293T cells and filter through 0.45 μm PES filter. The lentivirus were aliquoted and frozen or concentrated (skip to step 3). To aliquot, multiple aliquots of lentivirus were made ranging from few μl to 5 ml and depends upon the scale of transduction. For large scale production, mastermix of transfection mix were used for multiple petri dish. For larger production, the lentivirus aliquots of 5 ml were made in 15 ml tubes. Small aliquots of lentivirus media 50-200 μl were made for lentiviral titration. The Lentiviral stocks were then stored in minus 80° C. Freezer.

### Process for Concentration of the Lentivirus Library by Ultracentrifugation

Filter-sterile PBS/20% sucrose was prepared for the next day. After 48 hours, take the supernatant was taken. Spin the supernatant at 3,000 rpm for 5 min to preclear and filter on 0.45 μm. The volume of supernatant can be 14 ml pooled from 2 100 mm petri dishes. For Q-PCR, 20 U/ml of DNAaseI was added. 10 ml of filter-sterile PBS/20% sucrose was added to the bottom of the tubes and the supernatant was gently added to ultracentrifuge tubes on top of the sucrose pad. The supernatant and the sucrose should not be mixed. The ultracentrifuge was run at 35,000 rpm for 2 hours at 4° C. (Note: Ultracentrifugation is very sensitive for the balance of the tubes. The weight of the tubes should be exactly same.) After 2 hours, once the pellet is shown up, the pellet was pipetted gently. The supernatant and PBS-sucrose supernatant were discarded into 10% bleach. The viral pellet was then resuspended in 1 ml of complete DMEM (10% FBS+P/S)

### Titer Lentivirus Stock

### (1) Counting and Seeding of Cells for Transfection

LentiX293T cells were cultured in 100 mm petri dish in 10 ml complete DMEM media (DMEM+10% Fetal Calf Serum) and incubated CO2 incubator at 37° C. with 5% CO2 according to SOP2.x. The cells reaching 80%-90% confluent were ready for transfection. Spent DMEM media from the petri dish is aspirated out or discarded manually to the discard flask. The cells were trypsinized with 1.5 ml of 0.25% Trypsin solution according to SOP2.x Cells from the last step were resuspended om 3 ml of complete DMEM media. Cells were counted according to SOP2.x. 3 million cells were seeded in a 100 mm new petri dish having 10 ml complete DMEM media. Cells reaching 80-90% confluent were ready for the next passage. From the remaining cells, 0.5 million cells in 500 μl of complete DMEM media (DMEM+10% FBS) in a 15 ml tube were added to 4.5 ml of complete DMEM media. Cells (100 μl) were added to each well of the 96 well plate. The cells are 10,000/well.

### (2) Transduction of Cells

The cells were ready for transduction after 24 hours of seeding of the cells in the 96 well plate. A small aliquot of Lentivirus was thawed on ice and was used to measure the lentiviral titer.

Preparation of serial dilution of Lentivirus according to Table 1.

TABLE 1
Serial Dilution of Lentivirus

| Ratio | Lentivirus (μl) | DMEM (μl) |
|---|---|---|
| 1:10 | 50 | 450 |
| 1:50 | 100 (1:10) | 400 |
| 1:250 | 100 (1:50) | 400 |
| 1:1000 | 150 (1:250) | 450 |
| 1:5000 | 100 (1:1000) | 400 |
| 1:25,000 | 100 (1:5000) | 400 |
| 1:100,000 | 150 (1:25,000) | 450 |
| 1:500,000 | 100 (1:100,000) | 400 |
| 1:2,500,000 | 100 (1:500,000) | 400 |

The media was replaced with serial dilution of the Lentivirus (100 μl) and transduced for 4 hours. This was done in triplicate. After 24 hours, 100 μl of complete DMEM media with Puromycin (3 μg/ml) was added to bring a total volume of 200 μl and a final puromycin concentration of 1.5 μg/ml. Plates were then incubated at 37° C. for 120 hours in a CO2 incubator with 5% CO2. The cells were observed under microscope and the colonies in the highest dilution with colonies is considered to calculate the Infective units/ml (IFU). IFU/ml was calculated as below:

$$IFU / ml = number of colonies \times dilution factor \times 10.$$

Average the Triplicates were Averaged.

### (3) Freeze Concentrated Stocks

Stocks were frozen in 1.5 ml or 2 ml Cryovials which are labeled with the name of virus, date, initials of investigator, and titer. The lentiviral stocks were stored in minus 80 degrees Celsius Freezer.

### Example 2: Designing and Validating a Specific Fluorescent Assay for a Molecular Function or Cell Process

Verification of Functional Assay with Positive and Negative Controls.

A fluorescent assay of sequence-based assay was validated. This can be done by separate approaches either using fluorescent reporters, fluorescent dyes, or by immunostaining with an antibody. This example describes an immunostaining based example of phospho-Her2 in cells.

In step 4-6, cells were engineered, and the assay was tested for the specific assay using controls. Controls, including variants and generated using standard approaches and lentiviral plasmids encoding the controls were transduced into the reporter cell line. The reporter or immunostaining activity of the controls were measured by Flow cytometry, Fluorescence microscopy, and/or with a fluorimeter. Alternatively, a sequence-based reporter can be used. Controls include no Erbb2, the wild type Erbb2, and well-characterized variants with loss of function, gain of function, drug resistance, or drug susceptible variants. In step 6, conditions were changed to maximize the dynamic range of the reporter signal using controls. Additional details are listed below:

### Cell Culture Maintenance

HEK-293T cells expressing the TET-On 3G element HEK293T cells were used to generate cell lines for all experiments Cells were maintained and all experiments performed in Dulbecco's Modified Eagle medium (DMEM), 25 mM glucose, 1 mM sodium pyruvate (Thermo Fisher Scientific, #11-995-081) supplemented with 10% fetal bovine serum (Cytiva, #SH30396.03HI), 20 mM HEPES (Sigma Aldrich, #H3375), 60 mg/L penicillin G (Gold Bio, #P-304-100), and 100 mg/L streptomycin sulfate (Gold Bio, #S-150-50).

Transduction of Controls into Cell Lines

Cells transduced at a MOI 0.1 in the presence of 10 μg/mL polybrene (Millipore, TR-1003-G). Cells were poison selected in media supplemented with 2 μg/mL puromycin (Gold Bio, #P-600-100) for 7 days. Cells were frozen down in aliquots of 3E6/cryovial and stored in liquid nitrogen. For individual control assays, 1 vial of cells were thawed, then expanded for 1 passage prior to performing the assay. Maximum passage number for all experiments was passage 3.

For each expression construct or controls, 0.6×10⁶ Lenti-X 293T cells were plated into a well of a 6-well plate and cultured for 24 h. Each well was transiently transfected with 1.1 μg of a GML plasmid library, 1.1 μg psPAX2, and 0.6 μg pMD2.G using a 3 μL FuGENE HD: 1 μg DNA ratio. Representation of ~40 cells per clone. Each plate was transfected with 10 μg of plasmid library (~1.38×10⁶ copies of plasmid DNA per clone), 9.5 μg psPAX2, 5.2 μg pMD2.G using FuGENE as above. Media was changed 24 h post-transfection. Supernatant containing virus was harvested ~72 h post-transfection, filtered through a 0.45 μM filter, aliquoted, and stored in a freezer. Virus aliquots were thawed on ice and viral titers were measured with Lenti-X GOSTIX (Takara).

### Individual Control Experiments

1E6 cells were plated into 100 mm plates. 24 h after plating variant expression was stimulated by treatment of cells with 25 ng/ml doxycycline (Thermo Fisher Scientific, #BP25535) for 72 h. As the half-life of doxycycline in media is 24 h, 12.5 ng/ml of doxycycline was spiked into media every 24 h to maintain the 25 ng/mL concentration through-out variant induction. After 72 h, cells were harvested by trypsinization then fixed in 4% formaldehyde for 15 mins. Cells were then washed in 1×PBS before being permeabilized in 90% methanol (Thermo Fisher Scientific, BP1105-1) for 16 h. Cells were then washed in 1×PBS before being transferred to the blocking buffer (1×PBS supplemented with 1% Bovine Serum Albumin (Rockland Immunochemicals, #BSA-50) and 5 mM EDTA (Thermo Fisher Scientific, 15575020)) and stored at 4° C. until immunostaining.

### Example 3: High Throughput Screening Assay of GML

### Cell Line Production for Pooled Library

Cells were transduced with plasmid library lentivirus in the presence of 10 μg/mL polybrene at a MOI 0.1 with an expected yield of 20 cells transduced per clone. Cells were then poison selected with media supplemented with 2 μg/mL puromycin for 7 days. Cells were frozen down in aliquots of 5E6/cryovial and stored in liquid nitrogen. For GigaAssays, three vials (15E6 cells) were thawed, then expanded for 1 passage prior to performing the GigaAssay. Maximum passage number was 3-4

### High Throughput Screening Assay

For the GigaAssay, 40 million cells were plated on Day 1 for a final cell harvest of ~400-500 million cells on Day 5. Cells were plated at a density of 2 million cells/150 mm plate in 20 mL media with 20 plates per GigaAssay. 24 h after plating variant expression was stimulated by treatment of cells with 100 ng/mL doxycycline (Thermo Fisher Scientific, #BP25535) for 72 h. 50 ng/mL of doxycycline was spiked into media every 24 h to maintain the 100 ng/ml concentration through-out variant induction. After 72 h, cells were harvested by trypsinization then fixed in 4% formaldehyde for 15 mins. Cells were then washed in 1×PBS before being transferred to the blocking buffer (1×PBS supplemented with 1% Bovine Serum Albumin (Rockland Immunochemicals, #BSA-50) and 5 mM EDTA (Thermo Fisher Scientific, 15575020)) and stored at 4° C. until immunostaining.

### Immunostaining

Cells fixed in 4% formaldehyde for 15 min. Cells were then washed in PBS before being permeabilized in 90% methanol (Thermo Fisher Scientific) for 16 h. Cells were then washed in PBS, before being transferred to blocking buffer (PBS with 1% Bovine Serum Albumin (Rockland Immunochemicals) and 5 mM EDTA (Thermo Fisher Scientific)) and stored at 4° C. until immunostaining. Cells in blocking buffer were incubated with dilutions of monoclonal antibodies raised against human Her2 (rabbit mAb, Cell Signaling) and (p) Her2 (Tyr1248) (mouse mAb, Thermo Fisher Scientific) for 1 h at 4° C. Cells were washed in blocking buffer 3x times then incubated with secondary antibodies conjugated to different fluorophores for 1 h at 4° C. For individual control assays the following secondary antibodies were used; Rabbit IgG (H+L) Cross-Adsorbed, Alexa Fluor 647 conjugate (for Her2) (Fisher Scientific) and Mouse IgG (H+L) Cross-Adsorbed, Alexa Fluor 488 conjugate (for pHer2) (Thermo Fisher Scientific). For the GigaAssay the following secondary antibodies were used; Rabbit IgG (H+L) Cross-Adsorbed, PE conjugate (for Her2) (Fisher Scientific) and Mouse IgG (H+L) Cross-Adsorbed, Alexa Fluor 647 conjugate (for pHer2) (Thermo Fisher Scientific) for 1 hr. Cells were washed 3x in blocking buffer then stored at 4° C. in blocking buffer until FACs analysis or sorting.

Sorting of Cells into Bins by Flow Cytometry

Individual cells and GigaAssay experiments had cells analyzed and sorted by flow cytometry. All analytical flow cytometry experiments were with a Sony SH800Z flow cytometer (SONY, Tokyo, Japan). At least 10,000 events were captured for each sample. Cells were first gated for high expression of Her2 (AF647) then their level of pHer2 expression compared (AF488). FACs data analysis for individual control experiments was performed using FlowJo (v. 10.8.1, Ashland, OR).

Sorting of cell populations for the GigaAssay was performed by the Stanford flow cytometry core on a FACsAria II (BD, Franklin Lakes, NJ). Cells were gated for high expression of Her2 (PE) then sorted in 4 bins according to increasing level of pHer2 expression (AF647). Cell populations were sorted based by percent of the total cell population with Bin 1=50% lowest pHer2 expressors, Bin 2 the next 20%, Bin 3 the next 20% and Bin 4 being the 10% cells expressing the highest level of pHer2. 5×10⁶ cells were sorted into each bin After sorting, cells were pelleted by centrifugation, washed 1× with PBS and pellets were stored at minus 80 degrees Celsius.

### Example 4: Targeted NGS Sequencing of Umi Barcodes in GDNA

### Next-Generation Sequencing of UMI-Barcoded Variant Libraries.

For next-generation sequencing, PCR primers were designed to flank the targeted UMI region of the variant library while adding staggered nucleotides on 5′ and 3′ ends of the targeted insert for increasing diversity in the first 18 cycles of Illumina sequencing along with Nextera Read Adaptor sequences. The 5′ primers were a pool of 10 individual primers (oHI-00210-oHI-00219), all targeting the same site, but each containing a different staggered nucleotide sequence between the PCR primer binding site and the Nextera Read 1 adaptor sequence overhang on the 5′ end. The 3′ primers were also a pool of 10 individual primers (oHI-00333-oHI-00342) each containing a different staggered nucleotide sequence between the PCR primer binding site and Nextera Read 2 adaptor sequence overhang on the 5′ end. gDNA extracted from sorted cell populations was split and PCR amplified in multiple reactions (not using more than 2.0 μg DNA per reaction) with NEBNext Ultra II Q5 Master Mix (New England Biolabs, #M0544L, Ipswich, PA) with previously mentioned PCR primer pools using the PCR protocol: initial denaturation 98° C. for 2 min.; 25 cycles of denaturation at 98° C. for 10 s, annealing at 60° C. for 10 s, extension at 72° C. for 20 s; final extension at 72° C. for 2 min.; and ending with a hold of 4° C..

The PCR product ranging in size from 203-221 bp was purified using SPRIselect Beads for Size Exclusion and Selection (Beckman, #B23317, Brea, CA) with 0.65× right-sided bead: sample ratio and 1.0× left-sided bead: sample ratio, eluted in 30 μl Buffer EB (Qiagen, Germantown, MD), and pooled for each sorted cell population. 5 μL of the pooled size-selected PCR product from the first PCR was used in a limited-cycle second PCR to add dual-indexes and the P5 and P7 sequences on 5′ and 3′ ends, respectively. The PCR primers (oHI-00350-oHI-00369) were a selection of 8 primers with different i5 indexes and 12 primers with different i7 indexes so they could be used to produce up to 96 different dual-index combinations depending on the number of sorted cell populations being sequenced.

PCR was performed using KAPA HiFi HotStart ReadyMix following a PCR protocol: initial denaturation 98° C. for 3 min.; 6 or 8 cycles of denaturation at 98° C. for 30 s, annealing at 55° C. for 30 s, extension at 72° C. for 30 s; final extension at 72° C. for 5 min; and ending with a hold of 4° C.. The PCR product ranging in size from 272-290 bp was purified using SPRIselect Beads for Size Exclusion and Selection (Beckman, Brea, CA) with 0.8× right-sided bead: sample ratio and 1.0× left-sided bead: sample ratio and eluted in 25 μl Buffer EB (Qiagen, Germantown, MD). Final constructed NGS libraries were quantified using a Qubit dsDNA HS Assay Kit (Thermo Fisher Scientific, Hampton, NH) and checked for the expected size using the DNA 7500 Kit for the Bioanalyzer 2100 (Agilent, #Santa Clara, CA). Sequencing was performed on a NextSeq 500.

Genomic DNA (gDNA) Isolation from the Cells

Genomic DNA is isolated from cells harvested in the Step 3m using a Genomic DNA isolation kit (Zymogen Cat No. D4068 or Quick-DNA™ Miniprep Plus Kit) following the manufacturer's protocol:

The cells collected in a 15 ml tube were pelleted by centrifugation at 1000 rpm for 5 min. The media was discarded, 1 ml of PBS was added, and the resuspended cells are transferred to the 1.5 ml microfuge. The tube was centrifuged at 3,000 rpm for 5 min and the PBS was discarded by aspiration. The cell pellet was resuspended in 200 μl of PBS. 200 μl BioFluid & Cell Buffer (Red) and 20 μl Proteinase K was added. The cell pellet and the reagents in the tube were mixed thoroughly or vortex 10-15 seconds and then incubate the tube at 55° C. for 10 minutes. 1 volume Genomic Binding Buffer was added to the digested sample and mixed thoroughly for 10-15 seconds. The DNA mixture was then transferred to a Zymo-Spin™ IIC-XLR Column in a Collection Tube. Centrifuge at ≥12,000×g for 1 min. The collection tube was with the flow through. 400 μl DNA Pre-Wash Buffer was added to the spin column in a new Collection Tube. Centrifuge at ≥12,000×g for 1 min. The collection tube was emptied, and 700 μl g-DNA Wash Buffer was added to the spin column, the spin column was then centrifuged at ≥ 12,000×g for 1 min and the collection tune was emptied after centrifugation. 200 μl g-DNA Wash Buffer was added to the spin column. Centrifuge at ≥12,000×g for 1 min. The collection tube with the flow through was discarded and the spin column was transferred to a clean microcentrifuge tube. ≥50 μl of DNA Elution Buffer was added directly on the matrix and incubated for 5 minutes at room temperature, followed by centrifugation at maximum speed for 1 min to elute the DNA.

The eluted DNA is quantified with a Nanodrop spectrophotometer.

The eluted DNA can be used immediately for molecular based applications or stored at minus 20 degree Celsius for screening and future use.

### Example 5: Bioinformatic Analysis of Screening Results to Produce a Mega-Map with Variant Classification

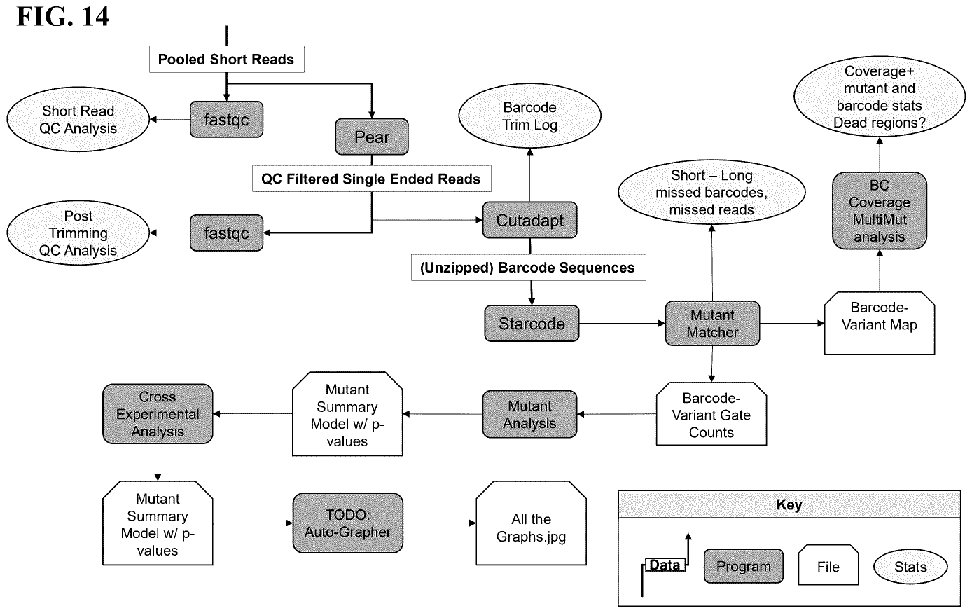

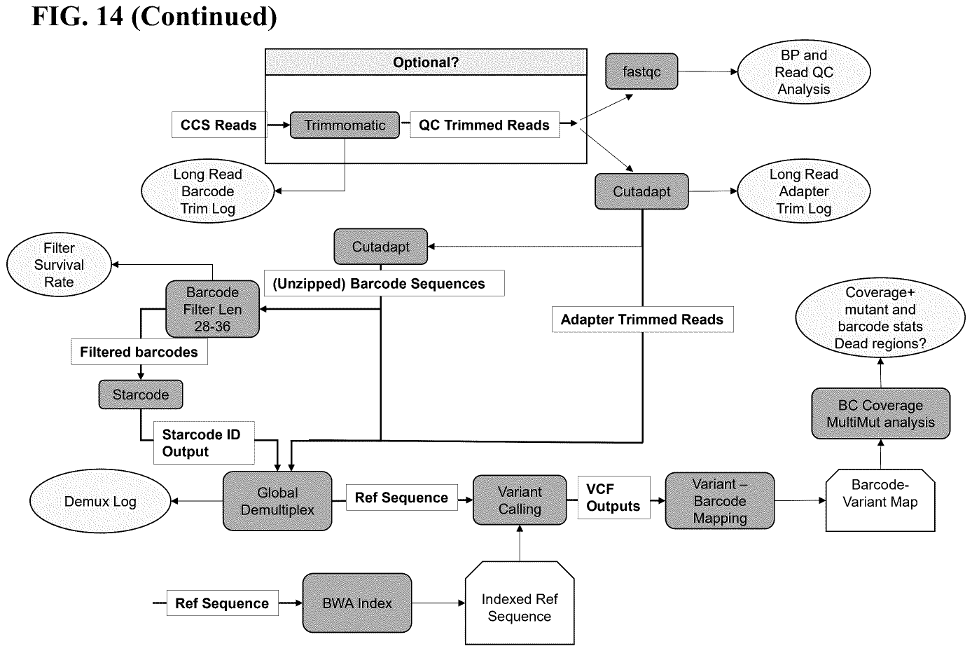

Each high throughput screening assay described herein used long-read NGS sequencing of a UMI-coded plasmid library and short read sequencing of gDNA isolated from flow-sorted bins. The pipeline is designed to use these data to call variants for each UMI coded cDNA and then calculate an activity for each UMI coded cDNA from UMI frequencies of short reads for each flow bin. The GigaAssay Bioinformatic pipeline is summarized in FIG. 14: Bioinformatics Pipeline. The goal of this pipeline is to produce a UMI barcode-mutant map, which creates an index between barcodes identified in the long-read library and the mutation in the library. This index is later used when flow-sorted short reads are produced and used for quantification of the barcodes to produce an activity score.

### QC Scanning and Read Trimming.

Long-read data for the library in a fastq file was analyzed for and trimmed of low-quality reads. This step is not necessary when using PacBio CCS reads. Reads with 4 base pair (BP) windows with average phred scores <=15 were identified and filtered using Trimmomatic. The following command was used on single-end read data, using 24 threads and producing a results file with all reads which passed the sliding window test. A log file was stored to trimmomaticLog.txt:

java -jar /data/fastpool/subvol-100-disk-
0/programs/Trimmomatic/Trimmomatic-0.39/trimmomatic-
0.39.jar SE -phred33 -threads 24 (IN FILE) (OUT FILE)
SLIDINGWINDOW:4:15 2>&1 | tee trimmomaticLog.txt

### Adapter Trimming

Reads were trimmed with Cutadapt such that the variant region and barcode are within the read with a 15 BP buffer before and after the variant region. Trimming reads produces space for sequence alignment and variant calling. Conserved regions of 12 BP (3′: CGTCAGATCCGC; 5′: GCGATCGCAGCG) were input into Cutadapt as 3′ and 5′ boundaries for removing excess BP. Cutadapt searched for these sequences using a default error tolerance that includes missense or indel errors (10%); this setting tolerates one error. When either or both 12 BP regions were located, the BPs in and before the 3′ region, and after the 5′ region were removed and copied to the output file; reads not trimmed or excised fragments are output to a separate file. Cutadapt detected, trimmed, and reversed those reads that were reverse complement reads in the output file, using--rc as a parameter in the command below:

~/.local/bin/cutadapt -g CGTCAGATCCGC...GCGATCGCAGCG
-j 24 --rc --untrimmed-output
missedAdapterTrimmedReads.fastq.gz -o (OUT FILE) (IN FILE)
2>&1 | tee adapterTrimLog.txt

### Barcode Extraction

Barcodes were extracted from reads to demultiplex reads by barcode while allowing for an error tolerance in the barcode. UMI barcodes (32 bp) were extracted from filtered reads with Cutadapt selecting a new 3′ region (12 bp) to trim extracting the barcode with the command:

~/.local/bin/cutadapt -g CGAGGAGATCTG -j 24 --untrimmed-
output missedBarcodeTrimmedReads.fastq.gz -o (OUT FILE) (IN
FILE) 2>&1 | tee barcodeTrimLog.txt

### Barcode Filtration

Rarely, during filtering the sequences align with the incorrect segment of the read and produce a barcode's size different than expected. Therefore, only barcodes between length 28-36 were selected with the Cutadapt awk command and included in the output file:

awk ‘BEGIN {FS = “\t” ; OFS = “\n”} {header = $0 ; getline seq ;
getline qheader ; getline qseq ; if (length(seq) >= 28 &&
length(seq) <= 36) {print header, seq, qheader, qseq}}’ < (IN
FILE) > (OUT FILE); awk ‘BEGIN {FS = “\t” ; OFS = “\n”}
{header = $0 ; getline seq ; getline qheader ; getline qseq ; if
(length(seq) < 28 ∥ length(seq) > 36) {print header, seq, qheader,
qseq}}’ < (IN FILE) > barcodeSizeFilteredOut.fastq

### Barcode Grouping

UMI Barcodes were grouped to contain UMIs with sequencing errors in the same group. Barcodes were clustered with Starcode using a Levenshtein distance of 2 with the following command:

/data/fastpool/subvol-100-disk-0/programs/starcode-
master/starcode -d 2 -t 24 --seq-id -i (IN FILE) -o (OUT FILE)

### Demultiplexing

Reads with the same UMI barcodes will occur in different samples such as different flow sorted bins. In order to call variant variants, reads that have the same barcode group must be isolated and grouped. This process was completed using a custom python script. This script uses outputs above to demultiplex reads based on their identified barcode group. Barcode groups where the master barcode length was not 32 were not demultiplexed. Reads were demultiplexed producing a directory and fastq file for each barcode group where the master barcode was length 32 with the following command is used:

python /data/fastpool/subvol-100-disk-
0/programs/custom/demuxer.py (B-4 OUTPUT) (B-3 OUTPUT)
(B-1 OUTPUT) (OUTPUT DIRECTORY)

### Variant Calling

A reference sequence was designated for sequence alignment and variant calling. The variant region (alongside the 15 BP buffer on each side) was placed into a fasta file for alignment Reads are aligned to the reference using BWA (http://bio-bwa.sourceforge.net/); the ref file was first indexed using the following command:
bwa index (IN FILE)

The file had its supporting index files generated in the same directory the file is present.

In order to map each barcode to a specific mutant, variants were called with a custom script called caller.py. The caller.py script parsed each barcode group's fastq into bwa mem, converted the output to a bam, used Samtools to sort it, Samtools mpileup it, and bcf tools to call unique variants. This produces a VCF derived from the reads associated with each barcode group.

CCS reads were reported to have incorrect indel call errors generated from incorrect sequencing. Indels were removed using callerNoIndel.py. Variants were called with the caller script, an indexed reference file, the demultiplex directory, and the required number of with the command:

python /data/fastpool/subvol-100-disk-
0/programs/custom/callerNoIndel.py (DIRECTORY FROM B-5)
(REF FILE FROM C-1) (NUMBER OF CORES TO USE)

Each fastq file in a subdirectory has a corresponding VCF file generated for use in variant interpretation.

### Indexing

Barcode hopping is frequently observed in sequencing results. Custom scripts quantify the number of barcode hopping event. The sorted bam were indexed in a subdirectory using the number of specified core with the following command to generate corresponding index file:

python /data/fastpool/subvol-100-disk-
0/programs/custom/Indexer.py (DIRECTORY FROM B-5)
(NUMBER OF CORES TO USE)

### Indel Calling

Insertion and deletion variants (indels) were identified with the program Nanocaller. The parameter settings that had the best performance were “--mode indels--mincov 3--sequencing pacbio--del_threshold 0.4--suppress_progress_bar--impute_indel_phase--enable_whatshap”; however, the parameters should be optimized for each library analyzed. These settings were captured in the program “IndelCaller.py” which was used to perform indel calling on all subdirectories:

Python /data/fastpool/subvol-100-disk-
0/programs/custom/IndelCaller.py (DIRECTORY FROM B-5)
(REF FILE FROM C-1) (NUMBER OF CORES TO USE)

### Mutant-Barcode Modeling

After variant calling, a mutant-barcode map was created for interpretation of the short-read sorting gates. A custom python script was used to filter reads based on QC and a required read depth per barcode. When working with CCS reads, QC 30, and depth of 1 is recommended. Instead, the ERBB2 library was processed with QC 0 minimum and depth of 1, with additional downstream filtration. This mutant-barcode map was created with the following command:

python MutantModelerOneFileIndelSupport.py (DIRECTORY
FROM B-5) (REF FILE FROM C-1) 30 1 (NUMBER OF CORES
TO USE) (TWIST FILE)

A csv file is output with every barcode group, each of the variants identified for that barcode group (and whether each variant was twist expected or not), and some QC statistics. All variant information required for the short-read interpretation and statistics was summarized and output to phenoModel.csv This flowchart figure describes the short and statistical part of the Long-Short read pipeline.

### Example 6: Use of Hifi DNA Assembly for Creating a GML

The steps below describe creation of a GML covering a variant region of 150 nucleotides with a coding sequence of a gene of interest using HiFi DNA assembly.

### Variant Assembly.

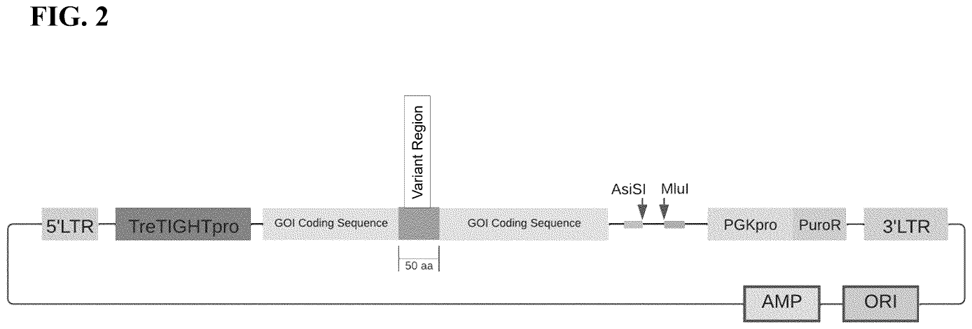

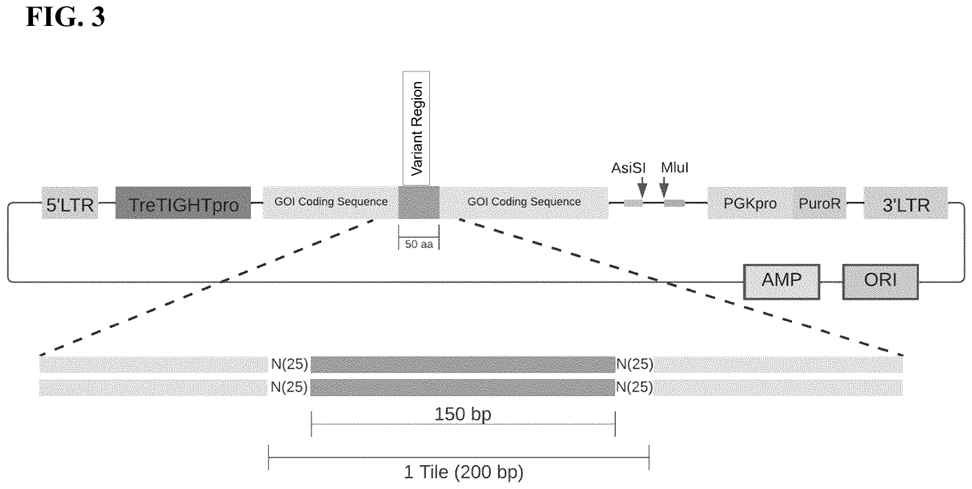

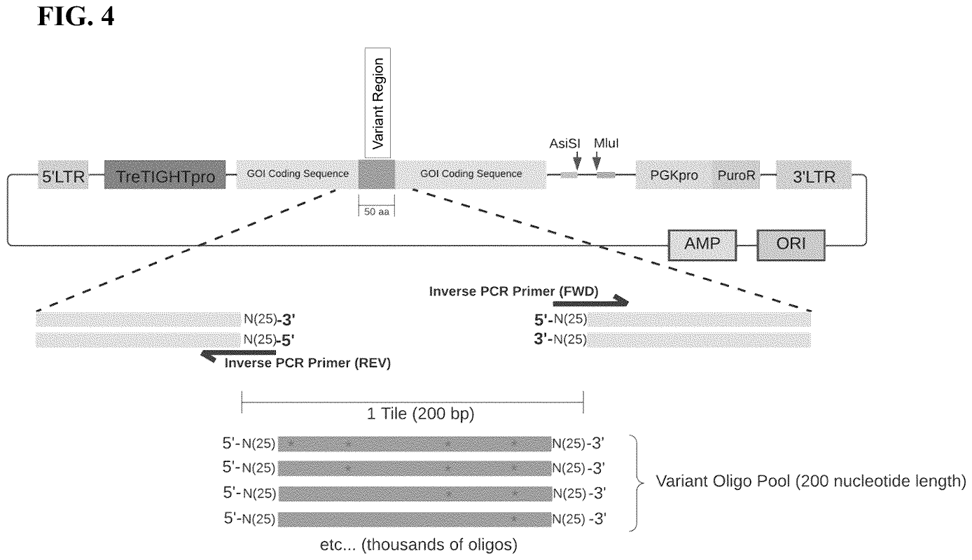

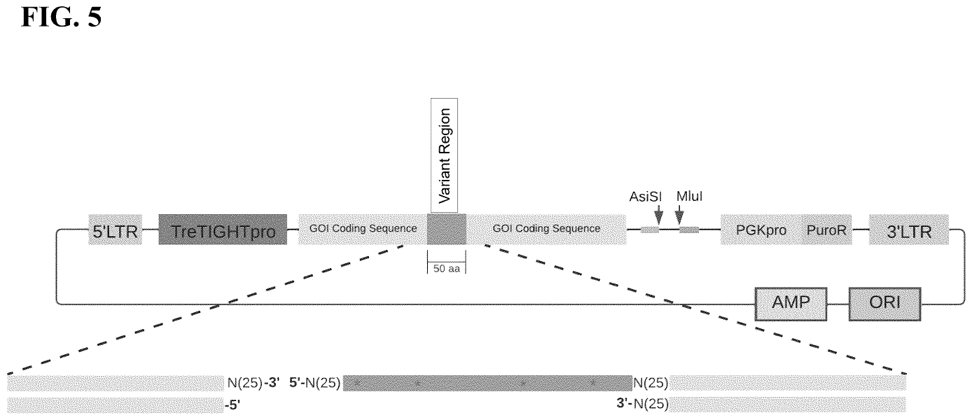

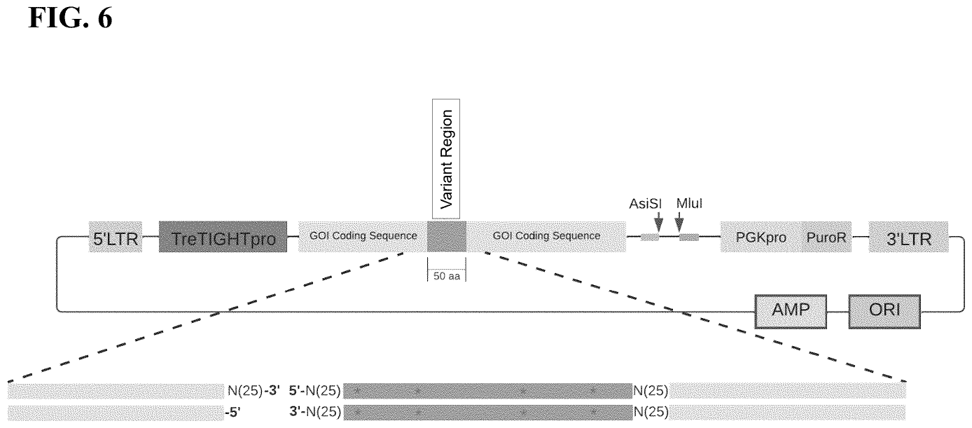

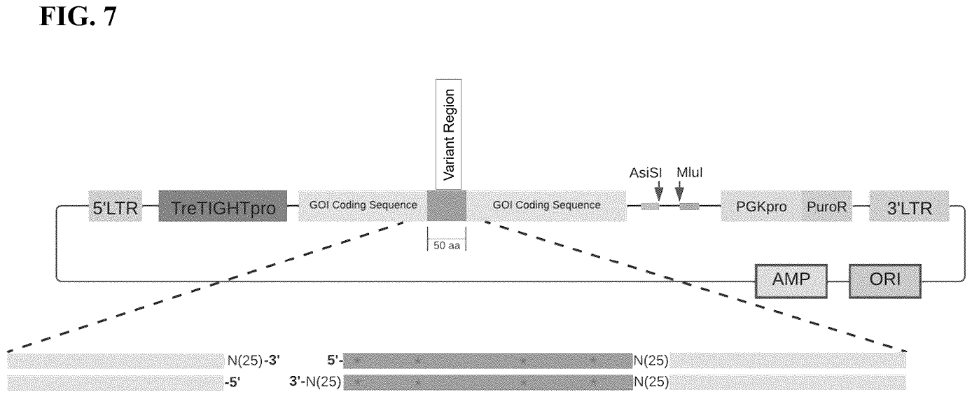

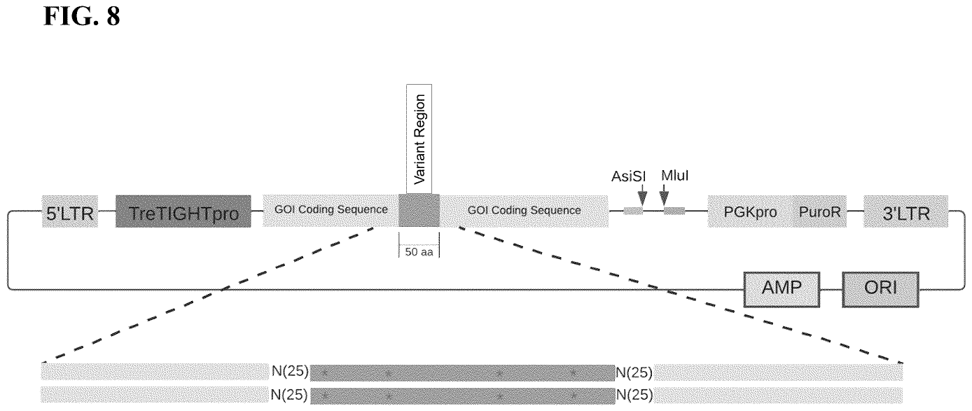

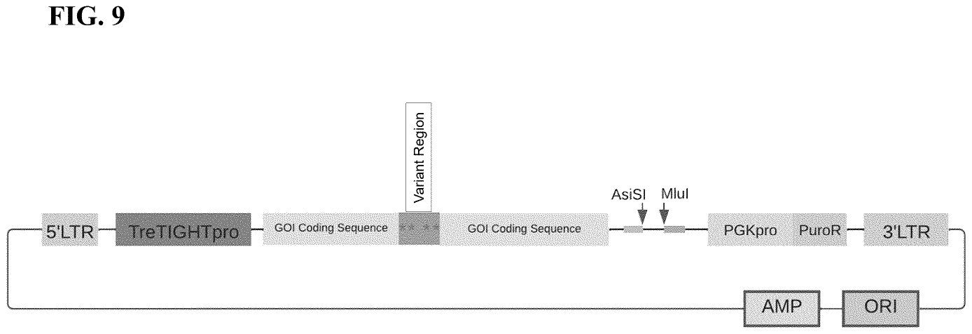

As shown in FIG. 2 a lentiviral expression vector contains a gene of interest (GOI) coding sequence with a variant region that corresponds to 50 amino acids of the coding sequence. The two sections near the AsiSI-MluI restriction sites in the 3′ UTR correspond to the UMI (Unique Molecular Identifier) insertion site which are covered in later steps during UMI Assembly. The variant region is 150 bp and 25 bp of overlapping DNA sequence flanking the variant region results in 1 tile of 200 bp (FIG. 3). FIG. 4 shows comparison of inverse PCR product and the variant oligo pool in preparation for HiFi DNA Assembly. Inverse PCR primers are designed so they include the 25 bp of sequence overlap with the oligo pool. The oligo pool consists of up to thousands of oligos containing desired mutations. T5 Exonuclease chews back dsDNA strands in 5′->3′ direction (FIG. 5). This exposes 3′ DNA strands for annealing to the complementary sequence found in the oligo pool (Note: This example is focusing on 1 oligo from the oligo pool.) The HiFi DNA Polymerase synthesizes DNA in a 5′->3′ direction, synthesizing the reverse strand of the variant oligo as well as filling in any gaps that occur after the annealing of DNA strands. DNA Ligase then seals the nicks to create a continuous DNA strand (FIG. 6). T5 Exonuclease then continues to digest exposed ends of DNA strands from 5′->3′ direction, exposing DNA for annealing complementary strands. (FIG. 7). The DNA Polymerase continues to synthesize DNA in 5′-3′ direction to fill in gaps, and DNA Ligase seals nicks to create continuous strands of DNA. The final result of a complete assembly is a scarless recombinant vector containing the variants of interest cloned into the variant region (FIG. 8). The result of a complete assembly is a scarless recombinant vector containing the variants of interest cloned into the variant region (FIG. 9).

HiFi DNA Assembly for GMLs that Require More than 1 Oligo Pool.

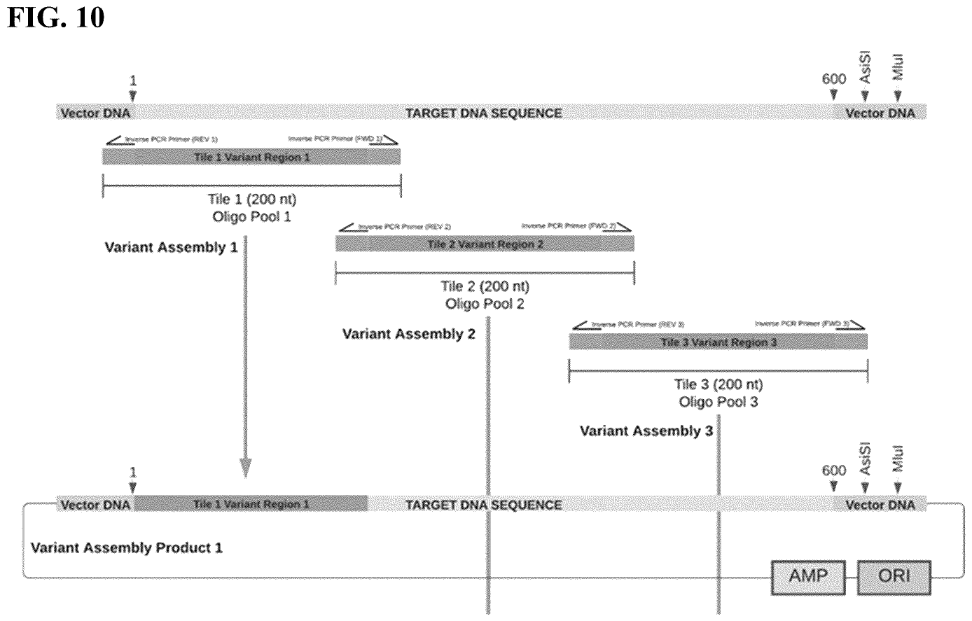

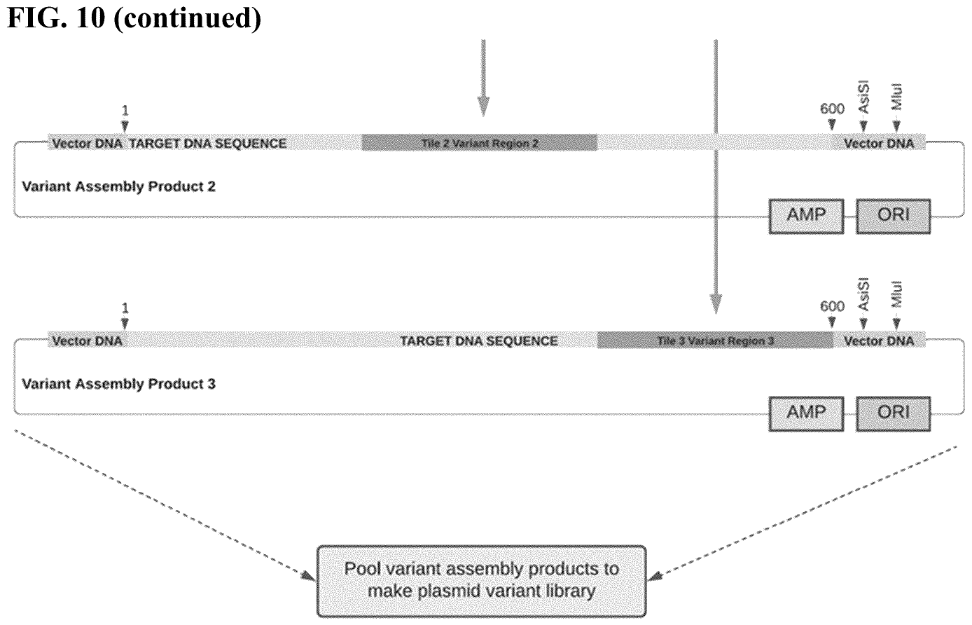

As shown in FIG. 10, the Target DNA Sequence (blue) is the variant region that is being targeted for the GML. The target DNA sequence is divided into tiles of 200 nucleotides. These tiles will correspond to 200-mer synthetic oligos for the variants of interest. Each tile consists of a variant region flanked by 25 nucleotides that overlap with the vector cloning site which also serve as Inverse PCR Primer binding sites for inverse PCR of the vector DNA. The tiles overlap so that the entire target region is covered. In this example the target DNA sequence is 600 bp long, covered by 3 overlapping tiles. Each tile is treated as separate Variant Assembly reactions into the expression vector using the same general strategy as described above. Variant Assembly results in 3 separate assembly product pools which will be combined to generate the plasmid variant library (without UMI). There are unique restriction sites in the 3′UTR of the vector (AsiSI and MluI). These sites are used to linearize the vector DNA to allow for HiFi DNA assembly to add a UMI to the 3′UTR with a ssDNA UMI oligo, resulting in a plasmid variant library with UMIs added to each plasmid DNA molecule of the library.

### UMI Assembly

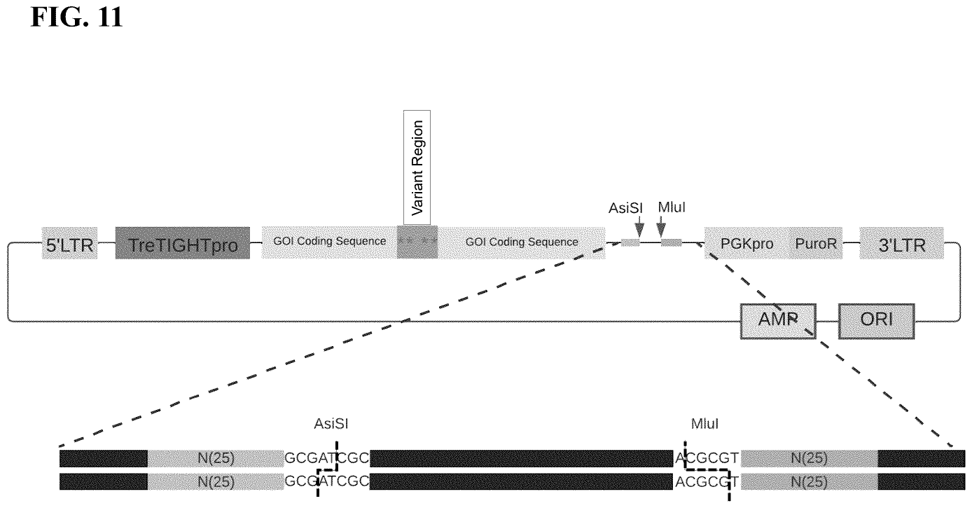

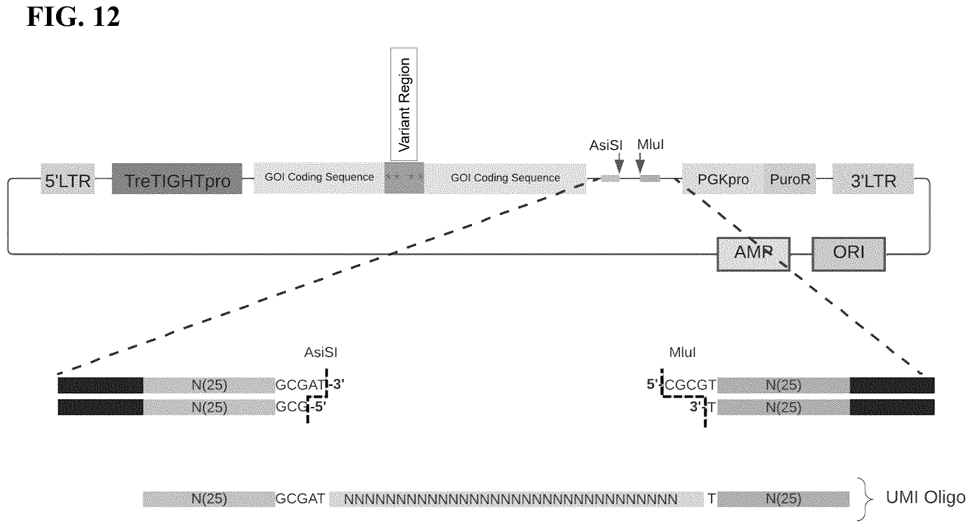

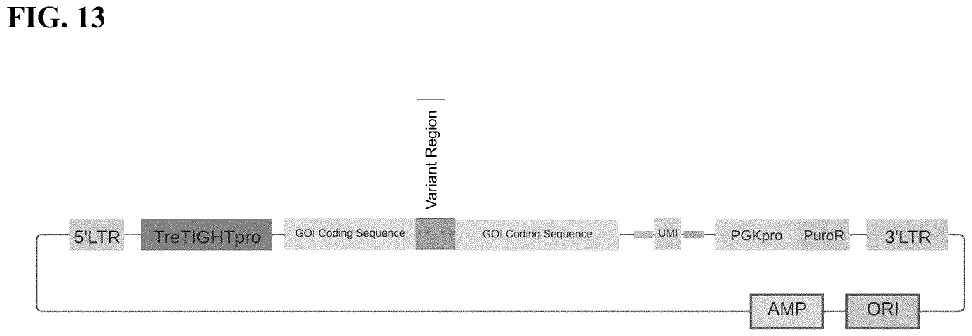

This step is like steps for Variant Assembly, except for UMI Assembly the variant plasmid library is linearized in the 3′ UTR using restriction enzymes. This linearized DNA is then used in a HiFi DNA Assembly reaction with a UMI oligo containing 25 nucleotides on 5′ and 3′ ends that overlap with 25 bp of DNA sequence on 5′ and 3′ ends of each linearized vector DNA molecule. The UMI oligo is designed to also include 32 degenerate nucleotides that serve as a unique molecular identifier for each molecule in the plasmid library. The 3′UTR UMI Assembly region (FIG. 11) contains two restriction sites, AsiSI and Mlul, which are used to linearize the variant plasmid library. The HiFi DNA Assembly reaction is performed using AsiSI-MluI linearized vector DNA and a UMI oligo that has 25 nucleotides of overlapping sequence with the cloning site (FIG. 12). The assembly reaction is carried out similar to FIGS. 5-8 above until assembly is complete. The final product of the UMI Assembly is a plasmid GML containing variants of interest with a unique molecular identifier for each plasmid DNA molecule in the library (FIG. 13).

Considerations for using HiFi DNA Assembly for GML Production

Before starting, the target DNA sequence and vector DNA sequence should not contain restriction sites that conflict with cloning steps, including the unique restriction sites in the 3′ UTR for UMI Assembly. Any restriction sites present in the DNA sequences should be removed in the library design.

The assembly steps require multiple rounds of electroporation depending on the desired clone count to achieve the library coverage that is desired (~200x coverage at a minimum). For example, if 200x coverage is need for 600 variants, 120,000 clones should be generated at each of these steps. The average cloning efficiency for Variant Assembly in combination with electroporation using Endura electrocompetent cells is: about 1×10\hat{ }8 CFU/ug or about 2×10\hat{ }6 CFU/ml for a tile of 200 bp and a vector size of about 11.2 kb. The average cloning efficiency for UMI Assembly in combination with electroporation using Endura electrocompetent cells is: about 1.7×10\hat{ }8 CFU/ug or about 3.4×10\hat{ }6 CFU/ml for a UMI oligo of 123 nucleotides and a vector size of ~11.2 kb.

Experimental Indexing of UMIs with Variant cDNAs by Long Read Sequencing of the Plasmid Library

### DNA Digest and Cleanup for PacBio Long-Read Sequencing

A restriction digest reaction is prepared to digest 10 μg plasmid-based gene variant library (Note: do not exceed 1 μg DNA per 50 μl restriction enzyme reaction):

Quantities are based on 1-50 μl reaction (scale accordingly for 10 μg DNA); the following Reaction Setup is used: 1 μg (plasmid-based gene variant library); to 50 μl Nuclease-free Water; 5.0 μl 10× CutSmart Buffer; 1.0 μl NheI-HF (30 U); 1.0 μl MluI-HF (30 U);

The reaction mixture is pipetted up/down 10× to mix, followed by centrifugation for 15 seconds to pull liquid down. After incubation at 37° C. for 1 hour, Gel Loading Dye, Purple (6X) (no SDS) is added to the entire reaction volume that to be loaded on the gel. The mixture is then again pipetted up/down 10× to mix, followed by brief centrifugation to pull liquid down.

Samples are loaded into their respective wells without puncturing the sides of the wells with the pipette tip; each well of a 6-well comb can hold up to 60 μl volume. The entire reaction volume is loaded onto agarose gel(s). 6 μl Benchtop kb DNA ladder is then loaded into an open lane of a well row. cover and electrodes are placed onto the unit to start electrophoresis with voltage set at 120 V. Electrophoresis is monitored periodically. Image of gel is acquired once the bands are clearly resolved and sizes can be determined using supplied photo viewer with smart phone or tablet.

Gel image is annotated in LucidChart (preferred) or PowerPoint. Gel image is labeled with experiment name, lane numbers, % agarose, and date. Gel gel extraction and cleanup of the desired DNA fragment are proceeded.

When using the NucleoSpin Gel Extraction kit, a heat block to 50° C. and 65° C. is set. A clean 1.5 ml microcentrifuge tube is used for each sample. Empty weight is added in the Weight Calculation table (in Tables section of the Benchling notebook entry). Gel is transferred to blue LED transilluminator. Blue LED light is turned on, a fresh razor blade is used to excise the desired DNA band.

The excised bands are placed into a microcentrifuge tube. Each tube corresponds to the excised band from each lane of the gel. Each tube with gel fragments are weighed and the total weight in the Gel is entered in the Weight Calculation table (in Tables section of the Benchling notebook entry).

The gel extraction is proceeded by the following steps: 2 volumes Buffer NT1 to is added to 1 volume of gel. Thus 200 μl Buffer NT1 is added to each 100 mg of gel. For >2% agarose gels, the volume of Buffer NT1 is doubled.

The mixture solution is heated at 50° C. for 10 min, or until the gel slice is completely dissolved. The solution is mixed by vortexing the tube every 2-3 min. during incubation. A NucleoSpin column is placed in a provided 2 ml collection tube. Up to 700 μl sample is loaded at a time to the column. (Note: per NucleoSpin manual, up to 200 mg of agarose can be dissolved with 400 μl of Buffer NT1 and loaded onto the column in one step. However, virtually unlimited amounts of gel can be loaded without clogging the column by increasing Buffer NT1 proportionally and adding multiple loading steps.) The column is spined for 30s at 11,000×g. Flow-through is discarded after spinning and the NucleoSpin column is placed back into the same collection tube. Remaining sample is loaded if necessary and repeat centrifugation step. The load steps are repeated for all the tubes corresponding to the excised DNA band for each plasmid library being processed so that all excised DNA from that sample is loaded onto the same column. If the total volume of restriction digest reaction volume plus loading dye for 10 μg plasmid DNA is 550 μl. 60-65 μl loaded into each lane of an agarose gel (using a 6-well comb) will result in 2 agarose gels, the first gel will get 5 lanes loaded with sample (60-65 μl per lane) and lane 6 will get the DNA ladder. The second agarose gel will get 4 lanes loaded with sample (60-65 μl per lane) and 1 open lane for DNA ladder. This will result in 9 tubes of excised DNA (each tube corresponding to each sample lane of the agarose gel). After adding Buffer NT1 and 10 minutes incubation at 50° C., those 9 tubes of excised DNA are loaded onto a single NucleoSpin spin column with up to 700 μl being loaded at a time.

Once all sample tubes of excised DNA corresponding to the same plasmid library are loaded onto the column, 650 μl of Buffer NT3 is added to the NucleoSpin column, followed by centrifugation for 30s at 11,000×g. After centrifugation, the flow-through is discarded. 650 μl of Buffer NT3 is then added to the NucleoSpin column, which is then centrifuged for 30s at 11,000×g. After centrifugation, flow-through is discarded. 650 μl of Buffer NT3 is to the NucleoSpin column, which is then centrifuged for 30s at 11,000×g. After centrifugation, flow-through is discarded without allowing the spin column to come into contact with the wash flowthrough. Outside of column is dried off with kimwipe, the content is transferred to new collection tube and centrifuged for additional 2 min. at 11,000×g to dry the column. The NucleoSpin spin column is placed into a fresh collection tube and place in a 65° C. heat block with lid open for 5 min to dry the column. The NucleoSpin column is then placed into clean 1.5 ml microcentrifuge tube. Desired volume (20 μl) (15-30 μl) of Buffer NE (room temperature) is added to center of the Nucleospin spin column membrane. The column is allowed to sit for 5 min at room temperature, followed by centrifugation for 1 min at 30×g, and 1 min. at 11,000×g. After centrifugation, another 20 μl of Buffer NE (room temperature) is added to center of the NucleoSpin spin column membrane. The column is then allowed to to sit for 1 min at room temperature, followed by centrifugation for 1 min at 30×g, and 1 min. at 11,000×g. After Centrifugation, another 20 μl of Buffer NE (room temperature) is added to center of the NucleoSpin spin column membrane. The column is then allowed to to sit for 1 min at room temperature, followed by centrifugation for 1 min at 30×g, and 1 min. at 11,000×g. After centrifugation, the spin-column is discarded. Eluted DNA is used in downstream applications. DNA is then quantified, the yield is calculated, and the data is recorded. The final cleanup is conducted using size-selection beads.

Before starting, either AMPure XP or SPRI Select size-selection beads is used. The size of the selected DNA fragments >2,000 bp. DNA fragments that are smaller than the target fragment size are removed. AMPure XP beads (if used) are placed at room temperature for 30 min prior to use. An adequate volume of fresh 80% molecular-grade ethanol is prepared for all samples being processed. Each sample requires 400 μl (not including dead volume). The sample in a 1.5 ml microcentrifuge tube in the process. If working with >8 samples and volumes <50 μl, the samples can be transferred to a 0.2 ml 96-well plate.

This procedure as stated is a 0.5× Bead: Sample ratio to bind all DNA fragments ≥~2000 bp, leaving smaller fragments in supernatant for removal.

The size-selection beads are vortexed until they are well dispersed, then 0.5 μl of well mixed size-selection beads per 1 μl of DNA sample is added. The entire volume is gently pipetted up and down 10 times to mix thoroughly. DNA fragments are bound to paramagnetic beads by incubating at RT for 15 mins.

The plate is placed on the magnetic stand for 2 minutes or until the supernatant has cleared. A pipette is used to remove and discard the supernatant which contains the unwanted size fragments and contaminants. With the tube still on the magnetic stand, 200 μl 80% ethanol is added to further remove contaminants. The plate is incubated on the magnetic stand at room temperature for 30 seconds, then the supernatant is removed and discarded. With the tube still on the magnetic stand, 200 μl 80% ethanol is added to further remove contaminants. The tube is incubated on the magnetic stand at room temperature for 30 sec., then the supernatant is removed and discarded. Sample is centrifuged in micro-centrifuge. after centrifugation, tube is placed back on magnetic stand and a P20 multichannel pipette is used with fine pipette tips to remove excess ethanol. Once all ethanol is removed and before the beads dry out, the plate is removed from the magnetic stand. 40 μL of elution buffer (10 mM Tris-HCl (pH 8.5) (i.e. Qiagen Buffer EB) is added to elute DNA with Gently pipetting up/down 10× to mix thoroughly. After incubation at room temperature without shaking for 5 minutes, tube is placed on magnetic stand for 2 minutes until the supernatant has been cleared. The entire volume is then transferred to a new screw cap microcentrifuge tube. Tips are changed between samples, or between rows or columns if using multichannel pipette.

DNA is then quantified, the yield is calculated, and the data is recorded.

40 ng of the DNA is run on a 1% agarose gel to ensure there is a single predominant band at the size expected. The DNA is then sent to sequencing core on dry ice for PacBio sequencing.

### Example 7: Application of the High Throughput Screening for Dominant Negative Biologics

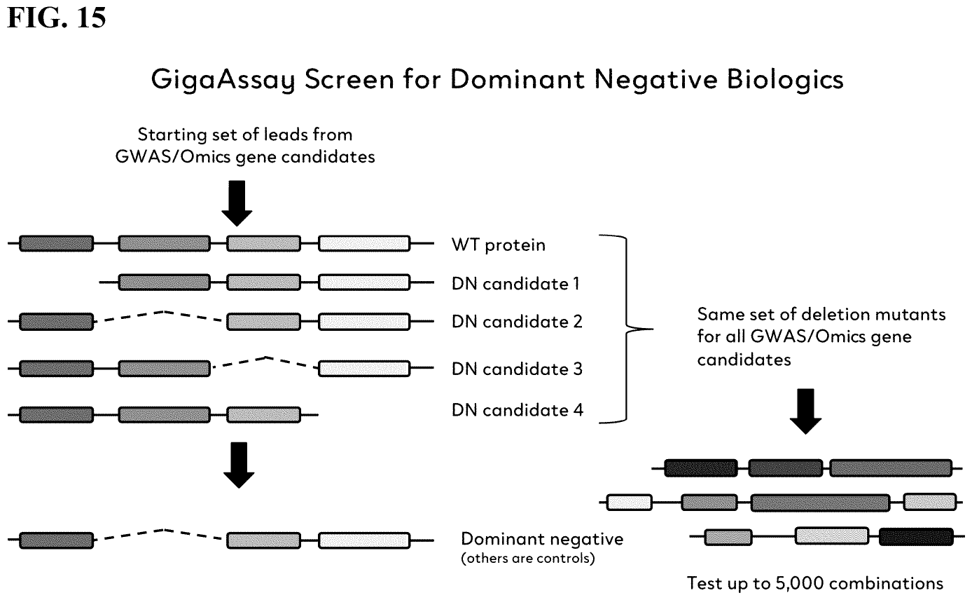

As shown in FIG. 15, to identify dominant negative biologics, a two-step strategy is used to analyze a set of leads or a subset of leads of gene coding regions to identify one or more lead therapeutics by experimentation. A high throughput screening assay described herein is used to identify dominant negative alleles that effect the pathological assay. As presently understood, the average human protein has 4.5 domains, where more than one is required for correct gene activity. A gene mutation library (GML) of variants of WT genes is generated with each variant deleting a different domain of the protein, while maintaining the correct reading frame (a dominant negative GML library for candidate genes prioritized by GWAS, polygenic risk scores (PRS) scores, or other Omics approaches). It is expected that one or more of these variants are function correctly. Since it has all the other domains, if competes for cell factors acts as a dominant negative allele. Alternatively, it multimerizes into a macromolecular complex with the wild type allele and acts as a point subunit 28755412, 19544283.

The particular high throughput screening assay chosen for these experiments is relevant to the pathological function that is to be treated, and thus is used to assess how a drug effects a molecular function or cell process, or both. Up to 5,000 combinations are tested with the high throughput screening assay described herein. A fluorescent readout in a cell, engineered cell, or both are generated as an outcome. The output from this first screen is used to identify dominant negative variants for most of the genes under test. One or more of the dominant negatives identified are explored as a new drug lead, acting like an antagonist for a particular pathway.

### Example 8: Application of the High Throughput Screening for Bispecific Biologics

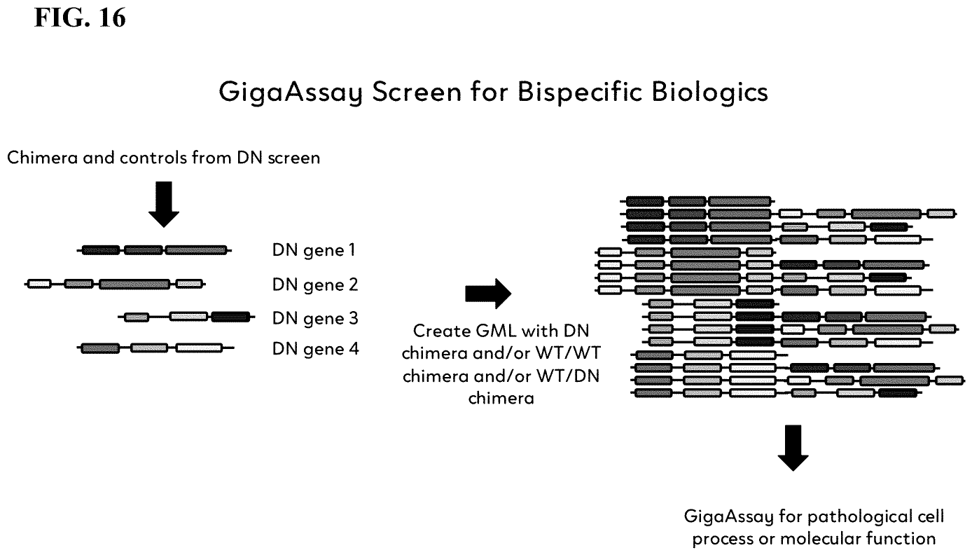

As shown in FIG. 16, to identify bispecific biologics, one or more of the identified dominant negative variants are used to make a new GML in which the genes with dominant negative variants are fused to wild type (WT) or DN variants versions of other genes to make a chimera library of all or nearly all variants using combinations of the starting genes and their variants. This GML is screened with a high throughput screening assay as described herein. Since each chimera may affect more than one reporter, multiple orthogonal high throughput screening assays are used to validate leads. The GML also includes control variants that are not DNs.

The high throughput screening assay described herein is used to identify inverse agonist or dominant negative versions of a gene, and thus one or both of them is to be developed into bispecific biologic drugs. Either can substitute for a neutralizing humanized monoclonal antibody therapy. This has the advantage of being a unique molecule and should not elicit an immune response like an antibody therapy as it has a small number of amino acid changes. These therapeutics can also be used to treat those patients that develop an immune response to an antibody-based therapy, as a more general substitute, or both. Examples include dominant negative PCSK9 for treating hypercholesterolemia or an inverse agonist of TNF as a substitute for Humira or related biosimilar drugs.

While preferred embodiments of the present disclosure have been shown and described herein, it will be obvious to those skilled in the art that such embodiments are provided by way of example only. Numerous variations, changes, and substitutions will now occur to those skilled in the art without departing from the method according to the present disclosure. It should be understood that various alternatives to the embodiments of the methods described herein may be employed in practicing the method of the present disclosure. It is intended that the following claims define the scope of the present disclosure and that methods and structures within the scope of these claims and their equivalents be covered thereby.

1. A method for identifying mutational effects on protein activity, the method comprising:
(a) obtaining a library comprising a plurality of cDNAs, wherein each cDNA in the plurality of cDNA encodes a unique variant(s) of a target protein, wherein each unique variant has one or more amino acid substitutions relative to the target protein;
(b) independently and individually incorporating one cDNA of the plurality of cDNAs into one plasmid, such that each cDNA of the plurality of cDNAs is individually and independently incorporated into its own plasmid and operably linked to a promoter on the plasmid, forming a plasmid cDNA library, and wherein each plasmid in the plasmid cDNA library further comprises a unique molecular identifier (UMI) or a barcode group;
(c) converting the plasmid cDNA library into a viral library comprising virions, wherein each virion comprises only one plasmid of the plasmid cDNA library, wherein the viral library can optionally be a lentiviral library;
(d) engineering a human or mammalian cell line to encode a receptor, optionally a surface-expressed receptor, operably linked to a reporter system such that the receptor and the reporter system can indicate if one of the unique variant(s) binds to and modulates a biological activity of the receptor, thereby forming an engineered human or mammalian cell line; and
(e) transducing cells of the engineered human or mammalian cell line of (d) with virions of the viral library of (c), such that each virion transduces a different cell, thereby forming a library of transduced cells.

2. The method of claim 1, wherein the method results in the expression of the unique variant of the target protein in a transduced cell of the engineered or mammalian cell line.

3. The method of claim 2, further comprising determining a measurement of a biological activity of the unique variant in the transduced cell of the engineered or mammalian cell line.

4. (canceled)

5. The method of claim 3, wherein the method further comprises sorting a transduced cell comprising the unique variant using a UMI or a barcode group by flow-cytometry.

6. The method of claim 5, comprising sorting a plurality, or all of the cells from the library of transduced cells resulting in the formation of a flow-sorted pool(s) of the UMI(s) or a flow-sorted pool(s) of the barcode group.

7. The method of claim 3, wherein the measurement of the biological activity comprises detecting binding of the unique variant(s) to the receptor operably linked the reporter system and determining an amount, an intensity, or both of a report from the reporter system, as measured by fluorescence microscopy, flow cytometry, or both.

8. The method of claim 5, further comprising comparing a distribution of the flow-sorted pools of the UMIs or the barcode groups of a single cell group that individually express the unique variant(s) to a distribution of flow-sorted pools of a single cell group expressing a wildtype target protein or a wildtype cDNA without the one or more amino acid substitutions.

9. (canceled)

10. The method of claim 1, wherein each UMI, or each barcode independently comprises about 12 nucleotides to about 32 nucleotides.

11. The method of claim 1, wherein the cells of the engineered human or mammalian cell line are transduced with the viral library at a multiplicity of infection (MOI) of at least 0.1 to at least 1.

12. The method of claim 11, wherein the MOI is 0.1.

13. The method of claim 11, wherein the MOI prevents double insertions of a virion into a single transduced cell or minimizes a transduction error rate.

14. The method of claim 1, wherein the cells of the engineered human or mammalian cell line are selected with a marker for lentiviral integration.

15. The method of claim 1, wherein the receptor and the reporter system are operably linked to an inducible-promoter.

16. The method of claim 15, wherein the inducible-promoter drives the expression of the receptor and the reporter system.

17. (canceled)

18. The method of claim 16, wherein the reporter system comprises a polynucleotide that encodes a fluorescent protein, and wherein the fluorescent protein is GFP.

19. The method of claim 1, wherein a single cell group is isolated based on the UMI using flow cytometry, and sorted into one or more bins, which comprise the cDNA(s) encoding the unique variant(s), an amplicon(s) thereof, or any combination thereof.

20. (canceled)

21. The method of claim 19, wherein the cDNA(s) encoding the unique variant(s), the amplicon(s) thereof, or any combination thereof are sequenced using a sequencing method.

22. The method of claim 21, wherein the sequencing method comprises next-generation sequencing, Sanger sequencing, whole genome sequencing, RNA sequencing or shotgun sequencing.

23-41. (canceled)
